(19) United States
(12) Patent Application Publication
(10) Pub. No.: US 2025/0384955 A1
(43) Pub. Date: Dec. 18, 2025

(54) SYSTEMS AND METHODS FOR IMPROVING LIVESTOCK PRODUCTION

(71) Applicant: Index Genetics, LLC, Stamford, CT (US)

(72) Inventors: Stewart BAUCK, Stamford, CT (US); W. Grant GREGORY, Stamford, CT (US); Ross HAVENS, Stamford, CT (US)

(73) Assignee: Index Genetics, LLC, Stamford, CT (US)

(21) Appl. No.: 19/179,794

(22) Filed: Apr. 15, 2025

Related U.S. Application Data
(60) Provisional application No. 63/634,159, filed on Apr. 15, 2024.
(60) Provisional application No. 63/769,986, filed on Mar. 11, 2025.

(51) Int. Cl. G16B 20/40; G16B 20/20

(52) U.S. Cl. CPC G16B 20/40; G16B 20/20

(57) ABSTRACT

A system and method for developing a personalized program for the identification and breeding of animals of superior genetic merit for a variety of traits.

### CROSS REFERENCE TO RELATED APPLICATIONS

This application claims benefit of and priority to U.S. Provisional Application No. 63/634,159 filed on Apr. 15, 2024, and to U.S. Provisional Application No. 63/769,986 filed on Mar. 11, 2025, which are incorporated by reference in their entirety.

### TECHNICAL FIELD OF THE INVENTION

The methods and systems of the present invention are generally directed to optimizing the selection and breeding of production animals for desired phenotypic traits. In one embodiment of a method according to the present invention, the production animals are cattle and, more specifically, include cows; replacement heifers; bulls; bovine progeny; and the like. The desired phenotypic traits include, but are not limited to disease resistance, such as resistance to Bovine Respiratory Disease (BRD); feed efficiency; reduction in methane emissions; tenderness; and other desired production traits. The invention further relates to methods and systems, including network-based processes, to manage the data relating to specific animals and herds of animals, veterinarian care, diagnostic and quality control data, breeding and management of livestock which have predictable traits, husbandry conditions, food safety information, and the like.

### BACKGROUND OF THE INVENTION

Significant improvements in animal performance, feed efficiency, carcass and meat quality, and other traits traditionally were made over the years through the application of standard animal breeding and selection techniques. For example, improvements in the milk production of dairy cows involved the studying of sire progenies and evaluating their milk production ratings (transmitting abilities) to guide further breeding. However, using standard breeding techniques may require years to evaluate the true genetic value since success depends upon the breeding of many cows and the subsequent birth of their offspring. For example, the females must be raised, bred, allowed to give birth and finally milked for a length of time to measure their phenotypic traits for milk production. Likewise, the females must be bred and allowed to give birth and the growth and performance of the offspring must be tested for phenotypic traits such as growth, reproductive efficiency, carcass composition and the like. Furthermore, selection based purely on phenotypic characteristics does not efficiently take into account genetic variability caused by complex gene action and interactions, and the effect of environmental and developmental variants. However, almost all animal production facilities can benefit from identifying and implementing improvements to production efficiency, which include improved proportional output of desired traits versus less desirable traits (for example lean meat produced compared to fat) as well as decreased production costs.

An animal production system may include any type of system or operation utilized in producing animals or animal-based products, such as milk and meat. Examples of production systems may include farms, such as dairy and beef farms, ranches, animal breeding facilities, and the like. Although animal production facilities may vary widely in scale, location, production purpose, and the like; almost all such facilities can benefit from identifying and implementing improvements to production efficiency. Such improvements can include anything that results in increased production results, improved proportional output of desired products versus less desirable products (e.g. lean vs. fat), and/or decreased production costs.

A producer such as a farmer, rancher, or the like generally benefits from maximizing the amount or quality of the product produced by an animal (for example, gallons of milk, pounds of meat, quality of meat, nutritional content of meat produced, amount of work including man-hours associated with production, health status, and the like) while reducing the cost for the inputs associated with that production. Exemplary inputs include, but are not limited to animal feed, animal facilities, animal production equipment, labor, veterinary care, medicine, and the like.

Animal feed generally encompasses compositions of a large variety of raw materials or ingredients. The ingredients can be selected to optimize any number of factors including the amount of any given nutrient or combination of nutrients in an animal feed product based upon the nutrient composition of the ingredients used.

Every variable input may further be associated with one or more effects of variation. For example, for almost every variable input, an increase in the amount of the variable input is associated with an increase in the cost of the variable input. For example, constructing additional facilities to increase production may be associated with building costs, financing costs, maintenance costs and the like. Further, the increase in the amount of the variable input is also associated with an increase in the benefit provided by the variable input. For example, the construction of the additional production facilities may be associated with an increase in the number of animals produced at the facility and the like.

Other efforts made to improve productivity and quality of animals produced include the application of various management practices including, but not limited to the use of standard breeding techniques, use of feed additives, animal hormonal implants, chemotherapeutics, and the like. However, such methodologies are non-inheritable and need to be applied differently in every production system and other drawbacks to some of these practices include significant regulatory resistance to the introduction and use of hormonal implants and the like. Other issues include those associated with delivering feed additives into animal feed rations in a feedlot accurately and on a customized basis at the time of feeding (see, inter alia, U.S. Pat. No. 4,733,971, issued Mar. 29, 1988; U.S. Pat. No. 4,889,433, issued Dec. 26, 1989; U.S. Pat. No. 4,815,042, issued Mar. 21, 1989; U.S. Pat. No. 5,219,224, issued Jun. 15, 1993; and U.S. Pat. No. 5,340,211, issued Aug. 23, 1994, U.S. Pat. No. 5,008,821, issued Apr. 16, 1991, all to Pratt and which contents are incorporated by reference in their entity). In a similar fashion, problems associated with keeping track of drug inventories; drugs administered to particular animals; and problems determining what drugs or combinations thereof should be administered, and in what dosages, to a particular animal diagnosed with a specific illness are discussed in, inter alia, U.S. Pat. No. 5,315,505, issued May 24, 1994 to Pratt.

Thus, producers have continually looked to additional methods for increasing productivity include methods for identifying and transmitting desired traits. One such method includes genetically evaluating cattle to enable breeders to more accurately select animals at both the phenotypic and the genetic level. For example, marker-assisted selection allows for a relatively easy and more efficient selection and breeding of farm animals, including cattle, for use with identifiable inheritable traits such as circulating leptin levels, feed intake, growth rate, body weight, carcass merit and carcass composition (see, for example, U.S. Pat. No. 7,947,444 to Moore; U.S. Pat. No. 7,897,749 to Khatib; U.S. Pat. No. 9,963,743 to Verschoor and Karrow; AU US2005233994 to Bauck et al; and U.S. Pat. No. 8,105,776 to Gill et al; the contents of which are all incorporated in their entirety).

A producer generally benefits from maximizing the amount or quality of the animal, especially if used as breeding stock, or product produced by the animal (for example, pounds of meat, quality of meat, resistance to common pathogens, and the like) while reducing the cost for the inputs associated with that production. Exemplary inputs may include animal feed, animal facilities, animal production equipment, labor, medicine, and the like.

To optimize a production system to evaluate and identify desired traits, such as tenderness; disease resistance, embryo lethality and the like, and introduce at least one trait into an animal and then produce progeny, a system for management and production is useful. One such system is the CARE Certified system which includes a set of sustainability standards that certifies participating producers are using best practices in animal husbandry and environmental stewardship. Other systems and programs are well known in the field or may be developed in other fields but applied to agriculture. For example, due to reoccurring concerns with food safety, producers and suppliers have examined systems to allow reliable traceability in supply chains and permit the fast retrieval of necessary information on food products. One possible way to address these challenges that is currently being explored is the application of blockchain technology (see, for example, Bosona, T.; Gebresenbet, G. The Role of Blockchain Technology in Promoting Traceability Systems in Agri-Food Production and Supply Chains. Sensors 2023, 23, 5342; the contents of which is incorporated in its entirety). Block chain technology also gives a consumer or other user assurance that the producer is in compliance with certification programs and other branded beef marketing schemes, such as but not limited to the Canadian Roundtable for Sustainable Beef.

Another well-known certification program is the Beef Quality Assurance Program whose mission is “to guide producers towards continuous improvement using science-based production practices that assure cattle well-being, beef quality and safety.” Members undergo training to become certified in good husbandry and management techniques with access to BQA programs that include best practices including cattle handling and transportation, management of facilities, and methods to protect herd health to ensure a commitment to food safety, cattle well-being, and beef quality.

One of ordinary skill in the art of animal breeding, production, and management will recognize that the CARE system, Beef Quality Assurance Program and Canadian Roundtable for Sustainable Beef are examples of a useful certification program, but any number of Certification programs can be useful in accordance with the present invention to provide a system and methods for optimizing and managing the selection of production animals, particularly cattle, for desired phenotypic traits. Participation in a certification program merely helps the consumer recognize the use of sustainable best practices all along the food production chain, including seedstock production; cow-calf production; stocker/backgrounding; and feedlot operations. Certification programs can further be used to provide assurance that a particular animal or product from that animal has a desired trait, such as BRD disease resistance, or has been raised in accordance with particular conditions such as in an antibiotic free environment or produced using humane ethical production methods. As consumers become more conscious of production issues, the demand for humane methods of production (see, for example, Front. Anim. Sci; 5 Sep. 2022; Hyland et al) and employing methods which reduce the impact on the environment has increased significantly.

### SUMMARY OF THE INVENTION

In accordance with the purpose(s) of this invention, as embodied and broadly described herein, one aspect of the present invention relates to systems and methods useful to manage improvements in animal production traits, especially those traits that are associated with economic importance including, but not limited to in economically important traits such as performance; feed efficiency; carcass and meat quality; reproductive performance or fertility; maternal ability; growth rate, body measurements, conformation, or structural soundness; longevity; carcass merit; and the like, whether the improvement is in the genotype, phenotype, or both of the animal produced and whether such improvement(s) is obtained through classical breeding methods or the use and identification of genetic markers associated with specific economically-important traits and subsequent breeding and production of animals having the identified genetic marker.

In one embodiment, the present invention provides systems and methods to selectively breed for desired characteristics in a production animal, such as a cow, a calf including replacement heifer, and bull, but one of ordinary skill will recognize that a similar system with its associated methods can be used for other production animals including, but not limited to swine; chickens and other poultry; donkeys; horses; goats; sheep and the like.

In another aspect of an invention in accordance with the present disclosure, it is a goal to propagate superior genetics through a defined pyramidal production scheme, including the testing of progeny for the desired trait and subsequent return of superior animals to the nucleus herd. A pyramidal production scheme refers generally to the selection of a limited number of animals, based on a desired trait for example, followed by the multiplication of those animals in the next generation resulting in a much larger number of animals, and then the production of the “production” animals in very large numbers in the final generation (see, for example, Van der Werf, Julius, An overview of animal breeding programs; Animal Breeding Use of New Technologies (This is a Post Graduate Foundation Publication)).

In another aspect of the present invention, improvements are contemplated to be in any trait which is of interest or is economically relevant and which can be identified and genetically introduced or passed to an offspring. Examples include but are not limited to the amount of or in the quality of the product produced by an animal, such as in the quantity of milk or meat produced or improvements in the quality of meat produced. Improvements may also be directed to increase the efficiency of production. For example, by identifying traits associated with feed efficiency that are heritable progeny can be produced that require less feed to produce the same amount of meat as a similar animal without the trait or traits. In a similar manner, one may breed animals that produce less methane even while consuming the same amount of feed as control animals without the trait.

In yet another aspect of the present invention, improvements are contemplated in the selection of animals for resistance to common diseases such as Bovine Respiratory Disease, so that fewer antibiotic treatments are required to produce a healthy animal.

In another aspect of the present invention, systems are contemplated to multiply superior breeding animals, such as an animal possessing and expressing a trait or traits of interest, using integrated, pyramidal production schemes in which genetic selection is made in the nucleus seedstock herds, and selected animals are used to populate multiplier herds to propagate superior genetics. In turn, breeding animals are selected from multiplier herds and used to populate commercial herds that breed and produce animals for consumption. A feature of the system is unique animal identification throughout the entire integrated or pyramidal production system, and accurate collection of phenotypic data from all animals using all available measurement systems, at all levels of the production system. This includes, but is not limited to individual animal feed intake, individual animal methane emission, meat tenderness, reproductive performance and fertility, including loss of embryos during any stage of pregnancy due to lethal defects, resistance to infection with any one of a number of infectious agents associated with Bovine Respiratory Disease, and the like. Data is then stored in a computer system, including pedigree information, and used to derive assessments, identify superior animals at all levels and move those superior animals up the pyramid for use in nucleus or multiplier herds.

In another aspect of the present invention, systems are contemplated and designed to obtain phenotypic information on animal performance using standard as well as novel data capture systems such as the C-lock Smartfeed system (Timothy DelCurto, Sam Wyffels, 4 Utilizing SmartFeed Pro and SuperSmart Feeders for applied beef cattle research, Journal of Animal Science, Volume 102, Issue Supplement_1, March 2024, Page 79), the Vytelle Sense system (Wells R S, Interrante S M, Sakkuma S S, Walker R S, Butler T J. Accuracy of the VYTELLE SENSE in-pen weighing positions. Appl Anim Sci. 2021; 37(5):626-34), the SenseHub ear tag (Hlimi A, El Otmani S, Elame F, Chentouf M, El Halimi R, Chebli Y. Application of Precision Technologies to Characterize Animal Behavior: A Review. Animals (Basel). 2024 Jan. 27; 14(3):416)), or similar systems. Such a system is useful for monitoring fever in an animal, including in cattle, providing an objective assessment of a clinical sign that can be used to assess progeny to produce, for example, an Expected Progeny Difference (Expected Progeny Differences Trait Definitions and Utilizing Percentile Tables. Sean Bessin and Darrh Bullock, Animal and Food Sciences, ASC-211: Expected Progeny Differences: Trait Definitions and Utilizing Percentile Tables) or Predicted Transmitting Ability or other similar recognized measure of genetic merit. Contemplated in this invention is assessment of combination of genetic merit for various synergistic or antagonistic traits of economic significance, to derive an index or weighted average of the genetic or economic merit of the animals, such as the Net Merit Index in dairy cattle (USDA AIP RESEARCH REPORT NM$8 (05-21) Net merit as a measure of lifetime profit: 2021 revision).

In another aspect of the present invention, systems are contemplated and designed to obtain phenotypic information on animal performance using standard as well as novel data capture systems such as GreenFeed, a turn-key system from C-Lock designed to measure gas fluxes of Methane (CH4), Carbon Dioxide (CO2), and optionally, Oxygen (O2), and Hydrogen (H2) from individual animals when they visit a feeder. The system requires a pelletized feed (<7 mm in diameter) to be used such as pelletized grass and alfalfa, and others using a concentrate mix. It is also possible to aggregate emissions data from individual animals and determine herd averages. The system is typically configured to offer a small amount of pelleted bait attractant to entice the animals to visit multiple times per day. The gas emissions data is logged when the animal visits to consume the feed, then processed allowing the user to easily access a summarized report of calculated fluxes.

The present invention also contemplates a system to capture biologic material, including DNA and RNA, for systematic evaluation through genotyping or sequencing. This includes individual animal identification and association of that animal ID with a corresponding sample ID such as is utilized in the Allflex Tissue Sampling Unit (U.S. Pat. No. 9,955,954B2 to Jean-Jacques M. Destoumieux Bruno M. Teychene). The sample collected may be of any type of biologic material from the animal including but not limited to hair samples with root bulb, semen, meat or other bodily tissue, saliva or oral swab including cheek swab, oocytes, cells extracted from a developing blastocyst, and so on. The sample ID, including bar code or other identifying feature, and corresponding animal ID are then entered into a data system. In a further embodiment, the tissue sample is subjected to genotyping or sequencing, including low pass sequencing and imputation, and the resulting genomic data is associated with the animal identification and sample identification and stored in the Data System. In a further embodiment, the animal ID and sample ID are associated with the individual animal phenotypic measures as described earlier and stored in the data system. The system also includes techniques such as the Genome Wide Association Study (Hayes B, Goddard M. Genome-wide association and genomic selection in animal breeding. Genome. 2010 November; 53(11):876-83) which are then used to examine association between genomic polymorphisms and trait variations. Those associations may then be used to identify superior and detrimental trait variations with the resulting trait associations further used to refine selection of superior breeding animals in future selection decisions in the pyramidal production system.

The present invention contemplates that genotyping can be done through a private laboratory or through a commercial provider such as Neogen. Neogen provides an array of services including testing for specific traits in production animals as well as genomic testing.

In another aspect of the present invention, enhanced reproductive technologies are used to efficiently and effectively multiply animals of superior genetic merit. These technologies include but are not limited to hormonal treatment to induce oocyte production in superior females, collection of oocytes, in-vitro fertilization of oocytes using conventional or sex-sorted semen (Reese S, Pirez M C, Steele H, Kölle S. The reproductive success of bovine sperm after sex-sorting: a meta-analysis. Sci Rep. 2021 Aug. 30; 11(1):17366), and transfer of fertilized oocytes and embryos to recipient females (Carlos R. Pinto, 2022, Overview of Embryo Transfer in Farm Animals, Merck Veterinary Manual). This system is contemplated to include oocyte collection from precocious, pre-pubertal females through direct aspiration from the ovaries (JIVET or Juvenile In-vitro Embryo Transfer) or similar methodologies (Armstrong D T, Kotaras P J, Earl C R. Advances in production of embryos in vitro from juvenile and prepubertal oocytes from the calf and lamb. Reprod Fertil Dev. 1997; 9(3):333-9) combined with fertilization from semen produced by precocious males, with the specific objective of multiplying genetics from superior animals and reducing the generation interval for more rapid genetic improvement.

In another aspect of the present invention, systems are contemplated for collection of biologic material from all animals in the production system at an early age, including but not limited to cells from developing blastocysts (Oliveira C S, Camargo L S A, da Silva M V G B, Saraiva N Z, Quintão C C, Machado M A. Embryo biopsies for genomic selection in tropical dairy cattle. Anim Reprod. 2023 Jul. 24; 20(2)). Using single nucleotide polymorphism (SNP) genotyping as well as other well-known sequencing technologies, SNPs and other genetic variants are identified and analyzed for their association to superior and/or detrimental production traits. One aspect contemplated to be within the present invention is the use of standardized genotyping arrays such as, but not limited to the Illumina Bovine SNP 50. Other sequencing techniques are well known to those of skill in the art and are considered to be within the scope of the present invention (M. Mahmoud, et al., Utility of long-read sequencing for All of Us. bioRxiv [Preprint]. 2023 Jan. 23; Qin D. Next-generation sequencing and its clinical application. Cancer Biol Med. 2019 February; 16(1):4-10). For example, the use of various sequencing techniques (short and long read) at varying levels of coverage of the genome to derive a more complete genome assembly of the animals is contemplated by the present disclosure. A further contemplated use is sequencing at low coverage, or low pass sequencing, following by imputation to full sequence using analytic methodologies known to those of skill in the art (Li J H, Mazur C A, Berisa T, Pickrell J K. Low-pass sequencing increases the power of GWAS and decreases measurement error of polygenic risk scores compared to genotyping arrays. Genome Res. 2021 April; 31(4):529-537).

In another aspect of the present invention, cloning technologies are used to efficiently and effectively multiply animals of superior genetic merit. It is contemplated that animals produced in nucleus, multiplier or commercial herds will be assessed for a variety of phenotypic measures and selected for cloning using currently available and accepted techniques (https://www.pnas.org/doi/epdf/10.1073/pnas.1501718112). It is anticipated that this will be done in beef but anyone familiar with agriculture will see that the same may be accomplished in sheep, dairy, pigs, chickens and the like

Yet another aspect of the present invention contemplates improvements in animal performance and specific traits using standard animal breeding and selection techniques. However, also contemplated is the use of genetic markers like single nucleotide polymorphisms (SNPs) that are associated with specific economically-important traits (especially traits with low heritability) to improve and optimize production animals, especially cattle, by identifying parent stock having the desired SNP and then using classical or enhanced breeding to establish the trait in progeny. In yet another embodiment, the invention contemplates the use of gene editing techniques to introduce a genetic sequence associated with the trait of interest into an animal.

Also contemplated to be within the present invention is a program comprising a system and methods to produce and manage production animals, especially cows and cattle, to optimize the improvements in animal performance and specific traits related thereto using, but not limited to somatic cell nuclear transfer (SCNT) by editing donor cells using CRISPR/Cas9 and other techniques commonly accepted as useful for gene editing.

Additional advantages of the invention will be set forth, in part, in the description which follows and, in part, will be obvious from the description, or may be learned by practice of the invention without undue experimentation. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiment(s) of the invention and, together with the description, serve to explain the principles of the invention.

FIG. 1 is an overview of a system in accordance with a method of the present invention whereby information from an individual animal and other inputs are provided, stored and analyzed with the results provided as an output to the participant using the method. A system such as Breedr is an example of a data management platform that encompasses elements of the system as outlined in FIG. 1

FIG. 2A is an overview of a pyramidal or integrated animal production system in accordance with a method of the present invention whereby superior animals are identified at the nucleus or Seedstock Herd level (eg. Nichols Farms), enhanced reproductive technologies are used to multiply superior genetics, genetic merit is assessed using traditional and genomic measures, and superior animals are moved to the next lower level of the pyramid for multiplication (eg. 2 Bar C Ranch) in Multiplier Herds and their progeny eventually moved to the Commercial Cow-Calf Herds.

FIG. 2B is an illustration of the number of cattle operations in the various levels of the Pyramidal Production System.

FIG. 2C is an overview of the flow of animals from the commercial cow calf sector in the pyramidal production system, including development and marketing of cattle through targeted feedlots (for example, AzTx Cattle Feeders and Gregory Feedlots). Phenotypic assessments are made at all levels and combined with genomic data and pedigree information from the pyramidal production system in a comprehensive manner, to derive accurate genetic merit as contemplated in FIG. 1.

FIG. 3 illustrates an Apex Herd Production Pyramid such as that embodied in Nichols Genetics production platform and which is used to produce replacement heifers and bulls to repopulate the Apex Herd, and to move in to the second tier of Cooperator Herds that will multiply the superior genetics identified, evaluated, and chosen to be incorporated in progeny.

FIG. 4 illustrates the Nichols Genetics Indexed Replacement Heifer and Bull Process as an embodiment for the production of replacement heifers and bulls in a method according to the present invention, wherein an outline of a typical DNA and phenotype collection process is provided.

FIG. 5A-5C illustrates a commercially-available greenfeed system such as that offered by C-Lock Inc., in Rapid City, South Dakota.

FIG. 6 illustrates a commercially available-feed intake system such as that offered as the Vytelle SENSE™ platform and system, located in Lenexa, Kansas, which collects data, including feed intake and weight measurements, to help assess individual animal performance to identify elite-performing animals and help guide informed genetic selection and marketing decisions.

FIG. 7 illustrates one embodiment of a water intake measurement system, such as the commercially available C-Lock SmartWater™ system (Rapid City, South Dakota).

FIGS. 8A-8B illustrate the SenseHub™ system (Merck Animal Health, Rahway, NJ) as one embodiment for a system useful for monitoring parameters in cow and calf operations such as body temperature and general health.

FIG. 9 illustrates an embodiment of a system useful for obtaining and monitoring animal weights such as the Vytelle SENSE™ system which, with its In-Pen Weighing Positions, is designed to collect individual animal weights in a non-intrusive way to allow for continuous monitoring of animal weights and growth rates.

### DETAILED DESCRIPTION OF THE INVENTION

### Definitions

It should be appreciated that this disclosure is not limited to the compositions and methods described herein as well as the experimental conditions described, as such it may vary. It is also to be understood that the terminology used herein is for the purpose of describing certain embodiments only, and is not intended to be limiting, since the scope of the present disclosure will be limited only by the appended claims. Unless defined otherwise, all technical and scientific terms used herein have the same meaning as commonly understood by one of ordinary skill in the art to which this disclosure belongs. Any compositions, methods, and materials similar or equivalent to those described herein can be used in the practice or testing of the present invention. All publications cited herein are incorporated by reference in their entirety.

The words “preferred” and “preferably” refer to embodiments of the invention that may afford certain benefits under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

Use of the term “about” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. It is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The use of the alternative (e.g., “or”) should be understood to mean either one, both, or any combination thereof of the alternatives.

The term “and/or” should be understood to mean either one, or both of the alternatives.

As used herein, the following terms have the meanings ascribed to them unless specified otherwise. In this disclosure, “comprises.” “comprising.” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. Throughout this specification, unless the context requires otherwise, the words “comprise”, “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment.” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “analyze” or variations thereof refers to determining the sequence, either directly (for example by actual sequencing) or indirectly (for example by the analysis of different fragment lengths following amplification and/or restriction enzyme cleavage). Typically, a method of the invention will be conducted by analyzing a sample obtained from the animal.

The term “animal” is used herein to include all vertebrate animals, including humans. It also includes an individual animal in all stages of development, including embryonic and fetal stages. As used herein, the term “production animals” is used interchangeably with “livestock animals” and “farm animals” and refers generally to animals raised primarily for meat, milk, or products such as wool and leather. For example, such animals include, but are not limited to, cattle (bovine), sheep (ovine), pigs (porcine or swine), poultry (avian), and the like. As used herein, the term “cow” or “cattle” is used generally to refer to an animal of bovine origin of any age. Interchangeable terms include “bovine”, “calf”, “steer”, “bull”, “heifer”, “cow” and the like. As used herein, the term “pig” is used generally to refer to an animal of porcine origin of any age. Interchangeable terms include “piglet”, “sow” and the like.

By the term “complementarity” or “complementary” is meant, for the purposes of the specification or claims, a sufficient number in the oligonucleotide of complementary base pairs in its sequence to interact specifically (hybridize) with the target nucleic acid sequence of the gene to be amplified or detected. As is known to those skilled in the art, a very high degree of complementarity is needed for specificity and sensitivity involving hybridization, although it need not be 100%. Thus, for example, an oligonucleotide that is identical in nucleotide sequence to an oligonucleotide disclosed herein, except for one base change or substitution, may function equivalently to the disclosed oligonucleotides. A “complementary DNA” or “cDNA” gene includes recombinant genes synthesized by reverse transcription of messenger RNA (“mRNA”).

The term “computer system” refers to the hardware means, software means and data storage means used to compile the data of the present invention. The minimum hardware means of computer-based systems of the invention may comprise a central processing unit (CPU), input means, output means, and data storage means. Desirably, a monitor is provided to visualize structure data. The data storage means may be RAM or other means for accessing computer readable media of the invention. Examples of such systems are microcomputer workstations available from Silicon Graphics Incorporated and Sun Microsystems running Unix based, Linux, Windows NT, XP or IBM OS/2 operating systems.

The term “conventional breeding techniques” and “traditional breeding techniques” are interchangeable terms and refer to breeding that employs processes which occur in nature, such as sexual reproduction. The product of conventional breeding emphasizes certain characteristics which are not new for the species. Cattle, particularly beef cattle, may be bred using traditional techniques to select for one or more desired traits including, but not limited to temperament, polledness, structural and udder soundness, disease and pest resistance, heat tolerance, “fleshing” ability, mothering ability, tenderness, feed efficiency, calving ease and the like.

The term “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

The term “database” refers to an organized collection of data, stored and accessed electronically, comprising of structured and unstructured data, and can be used to support a wide range of activities, including data storage, data analysis, and data management. There are many different types of databases, including relational databases, object-oriented databases, and NoSQL databases, and they can be used in a variety of settings, including business, scientific, and government organizations The information may be gathered from an individual or from an individual farm or feedlot or may be gathered from a collection of participants such as a breeding organization. Results of analysis of that data, information and content, and even communication with the participant and others is contemplated to be within the term “database.

“Denaturation” of a template molecule refers to the unfolding or other alteration of the structure of a template so as to make the template accessible to duplication. In the case of DNA, “denaturation” refers to the separation of the two complementary strands of the double helix, thereby creating two complementary, single stranded template molecules. “Denaturation” can be accomplished in any of a variety of ways, including by heat or by treatment of the DNA with a base or other denaturant.

A “detectable amount of product” refers to an amount of amplified nucleic acid that can be detected using standard laboratory tools. A “detectable marker” refers to a nucleotide analog that allows detection using visual or other means. For example, fluorescently labeled nucleotides can be incorporated into a nucleic acid during one or more steps of a cyclic polymerase-mediated reaction, thereby allowing the detection of the product of the reaction using, e.g. fluorescence microscopy or other fluorescence-detection instrumentation.

By the term “detectable moiety” is meant, for the purposes of the specification or claims, a label molecule (isotopic or non-isotopic) which is incorporated indirectly or directly into an oligonucleotide, wherein the label molecule facilitates the detection of the oligonucleotide in which it is incorporated, for example when the oligonucleotide is hybridized to amplified gene sequence. Thus, “detectable moiety” is used synonymously with “label molecule”. Synthesis of oligonucleotides can be accomplished by any one of several methods known to those skilled in the art. Label molecules, known to those skilled in the art as being useful for detection, include chemiluminescent or fluorescent molecules. Various fluorescent molecules are known in the art which are suitable for use to label a nucleic acid for the method of the present invention. The protocol for such incorporation may vary depending upon the fluorescent molecule used. Such protocols are known in the art for the respective fluorescent molecule.

By “detectably labeled” is meant that a fragment or an oligonucleotide contains a nucleotide that is radioactive, or that is substituted with a fluorophore, or that is substituted with some other molecular species that elicits a physical or chemical response that can be observed or detected by the naked eye or by means of instrumentation such as, without limitation, scintillation counters, calorimeters, UV spectrophotometers and the like. As used herein, a “label” or “tag” refers to a molecule that, when appended by, for example, without limitation, covalent bonding or hybridization, to another molecule, for example, also without limitation, a polynucleotide or polynucleotide fragment, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence, electrochemiluminescence, raman, calorimetric, hybridization protection assay, and mass spectrometry.

“DNA” refers to the polymeric form of deoxyribonucleotides (adenine, guanine, thymine, or cytosine) in either single stranded form, or as a double-stranded helix. This term refers only to the primary and secondary structure of the molecule and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in 5′ to 3′ direction along the nontranscribed strand of DNA (i.e., the strand having a sequence homologous to the mRNA).

“DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, and the like may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

Reference throughout this specification to “one embodiment,” “an embodiment,” “a particular embodiment,” “a related embodiment,” “a certain embodiment,” “an additional embodiment.” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “enhanced reproductive technologies” is used to describe methodologies to efficiently and effectively multiply animals of superior genetic merit. This includes but is not limited to hormonal treatment to induce oocyte production in superior females, collection of oocytes, in-vitro fertilization of oocytes using conventional or sex-sorted semen, and transfer of fertilized oocytes and embryos to recipient females. This system is contemplated to include oocyte collection from precocious, pre-pubertal females through direct aspiration from the ovaries (JIVET or Juvenile In-vitro Embryo Transfer) or similar methodologies (Armstrong D T, Kotaras P J, Earl C R. Advances in production of embryos in vitro from juvenile and prepubertal oocytes from the calf and lamb. Reprod Fertil Dev. 1997; 9(3):333-9) combined with fertilization from semen produced by precocious males, with the specific objective of multiplying genetics from superior animals and reducing the generation interval for more rapid genetic improvement. By the terms “enzymatically amplify” or “amplify” it is meant, for the purposes of the specification or claims, DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme QB replicase and a ribonucleic acid (RNA) sequence template attached to a probe complementary to the DNA to be copied which is used to make a DNA template for exponential production of complementary RNA; strand displacement amplification (SDA); QB replicase amplification (QBRA); self-sustained replication (3SR); and NASBA (nucleic acid sequence-based amplification), which can be performed on RNA or DNA as the nucleic acid sequence to be amplified.

The term “expected progeny difference (EPD)” uses an animal model database to create a genetic profile and contains information about an individual animal and its relatives to allow for the prediction of how future progeny of each animal are expected to perform relative to the progeny of other animals. EPDs are expressed in units of measure for the trait and can be expressed as positive or negative.

A “fragment” of a molecule such as a protein or nucleic acid is meant to refer to any portion of the amino acid or nucleotide genetic sequence.

The term “genetic evaluation” refers to an objective assessment of the merit of an individual animal and the likelihood that it will pass those traits on to any of its progeny. That assessment is derived using any number of commonly accepted computer-based methodologies. Several methodologies that are widely used include the Expected Progeny Difference (EPD) or the Expected Breeding Value (EBV) as commonly used in beef production, and the Predicted Transmitting Ability (PTA) as commonly used in dairy production (Animal Breeding and Genetics; Spangler, M. L., Ed.; Springer: New York, NY, USA, 2022).

As used herein, the term “genome” refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule). For example, it is known that the protein leptin is encoded by the ob (obese) gene and appears to be involved in the regulation of appetite, basal metabolism and fat deposition. In general, an animal's genetic characteristics, as defined by the nucleotide sequence of its genome, are known as its “genotype,” while the animal's physical traits are described as its phenotype.”

The term “gene editing” or “genome editing” refers to the precise, targeted change of a DNA sequence in a living cell. In production animals, such techniques could be used to produce an animal with a specific trait.

The term “genotyping” refers to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, a particular nucleotide in a genome may be an A in some individuals and a C in other individuals. Those individuals who have an A at the position have the A allele and those who have a C have the C allele. In a diploid organism the individual will have two copies of the sequence containing the polymorphic position so the individual may have an A allele and a C allele or alternatively two copies of the A allele or two copies of the C allele. Those individuals who have two copies of the C allele are homozygous for the C allele, those individuals who have two copies of the A allele are homozygous for the C allele, and those individuals who have one copy of each allele are heterozygous. The array may be designed to distinguish between each of these three possible outcomes. A polymorphic location may have two or more possible alleles, and the array may be designed to distinguish between all possible combinations.

By “heterozygous” or “heterozygous polymorphism” is meant that the two alleles of a diploid cell or organism at a given locus are different, that is, that they have a different nucleotide exchanged for the same nucleotide at the same place in their sequences.

By “homozygous” or “homozygous polymorphism” is meant that the two alleles of a diploid cell or organism at a given locus are identical, that is, that they have the same nucleotide for nucleotide exchange at the same place in their sequences.

By “hybridization” or “hybridizing.” as used herein, is meant the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequenced has a T, G, C or A, respectively. The hybridized fragment/oligonucleotide is called a “duplex.”

A “hybridization complex”, such as in a sandwich assay, means a complex of nucleic acid molecules including at least the target nucleic acid and a sensor probe. It may also include an anchor probe.

By “immobilized on a solid support” is meant that a fragment, primer or oligonucleotide is attached to a substance at a particular location in such a manner that the system containing the immobilized fragment, primer or oligonucleotide may be subjected to washing or other physical or chemical manipulation without being dislodged from that location. A number of solid supports and means of immobilizing nucleotide-containing molecules to them are known in the art; any of these supports and means may be used in the methods of this invention.

As used herein, the term “increased weight gain” means a biologically significant increase in weight gain above the mean of a given population.

The term “Index” is used to describe a combination of trait EPD's associated with appropriate economic valuation to provide a simple measure to achieve balanced selection and genetic progress. An example is the Nichols Family Farm Index which is derived from Feed Efficiency EPDs using data from the C-Lock SmartFeed bunk and SmartWeigh scale system which gathers individual feed intake on bulls and heifers. An average daily gain (ADG) EPD is calculated as the difference between the animals actual ADG and expected ADG based on size and growth of an animal on a feed test. The residual feed intake (RFI) EPD is the difference between an animal's actual intake and expected intake based on size and growth of an animal on a feed test. The Nichols Family Farm index combines the values along with appropriate weighting measures to derive a final index:

$$Index = 118 + 115 * adg\_epd - 48 * rfi\_epd$$

The Nichols Family Farm Index is one type of index, but it will be appreciated by one of skill in the art that an Index may contain any variety of phenotypic measures or genetic evaluations, with any number of appropriate economic weighting factors.

As used herein, the term “locus” or “loci” refers to the site of a gene on a chromosome. A single allele from each locus is inherited from each parent. Each animal's particular combination of alleles is referred to as its “genotype”. Where both alleles are identical, the individual is said to be homozygous for the trait controlled by that pair of alleles; where the alleles are different, the individual is said to be heterozygous for the trait.

A “melting temperature” is meant the temperature at which hybridized duplexes dehybridize and return to their single-stranded state. Likewise, hybridization will not occur in the first place between two oligonucleotides, or, herein, an oligonucleotide and a fragment, at temperatures above the melting temperature of the resulting duplex. It is presently advantageous that the difference in melting point temperatures of oligonucleotide-fragment duplexes of this invention be from about 1° C. to about 10° C. so as to be readily detectable.

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

“Polymerase chain reaction” or “PCR” refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to affect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

A “polymerase” is an enzyme that catalyzes the sequential addition of monomeric units to a polymeric chain, or links two or more monomeric units to initiate a polymeric chain. In advantageous embodiments of this invention, the “polymerase” will work by adding monomeric units whose identity is determined by, and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

A “polynucleotide” refers to a linear chain of nucleotides connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides liked by a phosphodiester backbone. A “modified polynucleotide” refers to a polynucleotide in which one or more natural nucleotides have been partially or substantially replaced with modified nucleotides.

The term “predicted transmitting abilities (PTA)” refers to a genetic evaluation tool that allows a comparison of two animals and provides estimates of an animal's genetic value for a given trait that will be passed to offspring. Examples of such traits include, but are not limited to milk yield, protein percent, fat percent, somatic cell score, and calving ease.

A “primer” is an oligonucleotide, the sequence of at least a portion of which is complementary to a segment of a template DNA which to be amplified or replicated. Typically, primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

“Probes” refer to oligonucleotides nucleic acid sequences of variable length, used in the detection of identical, similar, or complementary nucleic acid sequences by hybridization. An oligonucleotide sequence used as a detection probe may be labeled with a detectable moiety. Various labeling moieties are known in the art. Said moiety may, for example, either be a radioactive compound, a detectable enzyme (e.g. horse radish peroxidase (HRP)) or any other moiety capable of generating a detectable signal such as a calorimetric, fluorescent, chemiluminescent or electrochemiluminescent signal. The detectable moiety may be detected using known methods.

As used herein, the term “protein” refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

As used herein, the terms “traits”, “quality traits,” “production traits,” “genotype,” or “physical characteristics,” which may be used interchangeably, generally refer to advantageous properties of the animal resulting from genetics. In cattle, common traits include heavily muscling; high carcass yield for red meat; growth rate in the live animal; feed efficiency; and milk production. However, other quality traits are known which include, but are not limited to, the animal's genetic ability to metabolize energy, confer resistance to infection with a pathogen such as Bovine Respiratory Disease (BRD); decrease embryonic lethality; put on intramuscular fat; lay eggs; produce offspring; produce particular proteins in meat or milk; retain protein in milk; and the like. Physical characteristics include, but are not limited to, marbled or lean meat produced or may encompass body characteristics such as conformation that allows a cow to deliver offspring more easily and thus affect the number of live births.

The term “phenotype” is generally referred to as the animal's observable characteristics, determined by environmental influences and genetic makeup. In cattle, phenotype can take on qualitative or quantitative properties. An example of a phenotype in cattle relates to the presence or absence of horns.

The term “genotype” generally refers to the genetic code of an animal consisting of a pair of alleles that code for an inheritable trait or characteristic. The alleles represent differences in the DNA code that may or may not result in observable traits of the animal depending upon factors such as whether the traits are dominant or recessive. The term “homozygous” refers to an animal having two identical alleles of a particular gene, while “heterozygous” refers to an animal having two different alleles of a particular gene. For example, mating a homozygous bull with two horned alleles (the genotype is designated as pp to represent a recessive trait) with a homozygous cow with two polled alleles (designated as PP to represent a dominant trait) results in a calf with a genotype that is heterozygous for this trait (represented as Pp with one allele from the cow and the other from the bull) but its phenotype will be polled because the polled allele is dominant.

A “restriction enzyme” refers to an endonuclease (an enzyme that cleaves phosphodiester bonds within a polynucleotide chain) that cleaves DNA in response to a recognition site on the DNA. The recognition site (restriction site) consists of a specific sequence of nucleotides typically about 4-8 nucleotides long.

A “single nucleotide polymorphism” or “SNP” refers to polynucleotide that differs from another polynucleotide by a single nucleotide exchange. For example, without limitation, exchanging one A for one C, G, or T in the entire sequence of polynucleotide constitutes a SNP. Of course, it is possible to have more than one SNP in a particular polynucleotide. For example, at one locus in a polynucleotide, a C may be exchanged for a T, at another locus a G may be exchanged for an A, and so on. When referring to SNPs, the polynucleotide is most often DNA.

As used herein, a “template” refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such DNA strand may be single-stranded, or it may be part of a double-stranded DNA template. In applications of the present invention requiring repeated cycles of polymerization, e.g., the polymerase chain reaction (PCR), the template strand itself may become modified by incorporation of modified nucleotides, et still serve as a template for a polymerase to synthesize additional polynucleotides.

A “thermocyclic reaction” is a multi-step reaction wherein at least two steps are accomplished by changing the temperature of the reaction.

A “thermostable polymerase” refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include Taq, Tth, Pfu, Vent, deep vent, Ultima, and variations and derivatives thereof.

A “variance” is a difference in the nucleotide sequence among related polynucleotides. The difference may be the deletion of one or more nucleotides from the sequence of one polynucleotide compared to the sequence of a related polynucleotide, the addition of one or more nucleotides or the substitution of one nucleotide for another. The terms “mutation,” “polymorphism” and “variance” are used interchangeably herein. As used herein, the term “variance” in the singular is to be construed to include multiple variances; i.e., two or more nucleotide additions, deletions and/or substitutions in the same polynucleotide. A “point mutation” refers to a single substitution of one nucleotide for another. Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the resent invention, suitable methods and materials are described herein.

The term “input system” may comprise a computer mouse, keyboard, touch screen, spoken voice input, text inputs, scale inputs for weighing, EID tag, temperature probe, ear tag, and/or any other visual, tactile, movement-based, and auditory means, or any other means described herein.

The term “computing device” may comprise an electronic device such as a personal computer, tablet computer, smartphone, laptop, virtual reality device, or others.

The term “output system” may comprise a computer monitor, projector, printer, smartphone screen, speaker, LED display, or any other display system or other means described herein.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements. The description exemplifies illustrative embodiments. The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention. In several places throughout the application, guidance is provided through lists of examples, which examples can be used in various combinations. In each instance, the recited list serves only as a representative group and should not be interpreted as an exclusive list. All headings are for the convenience of the reader and should not be used to limit the meaning of the text that follows the heading, unless so specified. It is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in any appropriately detailed structure.

The present invention is generally directed to optimizing the production, evaluation, and selection of animals for at least one desired phenotypic trait. In one embodiment of the present invention, it is contemplated that the optimization, evaluation, and selection of animals is directed towards production animals and, more particularly, the invention is directed to cattle, including cows; calves; steer; heifers including replacement heifers; bulls; bovine progeny; and the like and encompassing an individual animal in all stages of development, including embryonic and fetal stages.

The present invention is also generally directed to optimizing the production of system that utilizes a pyramidal or integrated beef production system in which genetic selection is made in the nucleus seedstock herds, and selected animals are used to populate multiplier herds to propagate superior genetics. In turn breeding animals are selected from multiplier herds and used to populate commercial herds that breed and produce animals for consumption. A feature of the system is unique animal identification throughout the entire integrated or pyramidal production system, and accurate collection of phenotypic data from all animals using all available measurement systems, at all levels of the production system. This contemplates detailed phenotypic measurement of animals, as described previously, in the final stage of production as, for example, in a feedlot and associating the performance of those animals directly to the breeding animals that produced them, through a Data System. This would include but not be limited to individual animal feed intake, individual animal methane emission, meat tenderness, reproductive performance and fertility including loss of embryos during any stage of pregnancy due to lethal defects, resistance to infection with any one of a number of infectious agents associated with Bovine Respiratory Disease, and the like. Data is then stored in the Data System, including pedigree information, and used to derive assessments including genetic evaluation to derive EPD, identify superior animals at all levels and move those superior animals up the pyramid for use in nucleus or multiplier herds.

In one embodiment the present invention is useful in all manner of bovines, including but not limited to cows, calves, bulls and the like. However, the scope of the present invention also contemplates the use of other common production or farm animals in a method of the present invention. For example, the production animals may be pigs or swine; sheep; goats; poultry including chickens, geese, ducks, turkeys, guinea fowl, pigeons, ostriches, quail, pheasants; horses; donkeys; rabbits; honeybees; aquaculture and the like.

In another aspect of the present invention, enhanced reproductive technologies are used to efficiently and effectively multiply animals of superior genetic merit. These technologies include but are not limited to hormonal treatment to induce oocyte production in superior females, collection of oocytes, in-vitro fertilization of oocytes using conventional or sex-sorted semen (Reese S, Pirez M C, Steele H, Kölle S. The reproductive success of bovine sperm after sex-sorting: a meta-analysis. Sci Rep. 2021 Aug. 30; 11(1):17366), and transfer of fertilized oocytes and embryos to recipient females (Carlos R. Pinto, 2022, Overview of Embryo Transfer in Farm Animals, Merck Veterinary Manual). This system is contemplated to include oocyte collection from precocious, pre-pubertal females through direct aspiration from the ovaries (JIVET or Juvenile In-vitro Embryo Transfer) or similar methodologies (Armstrong D T, Kotaras P J, Earl C R. Advances in production of embryos in vitro from juvenile and prepubertal oocytes from the calf and lamb. Reprod Fertil Dev. 1997; 9(3):333-9) combined with fertilization from semen produced by precocious males, with the specific objective of multiplying genetics from superior animals and reducing the generation interval for more rapid genetic improvement.

Yet a further aspect of the present invention contemplates isolating and obtaining desired animals following identification of certain preferred traits so that these animals can be used as breeders to supplement a herd with a desired trait or traits. For example, using the system of the present invention, animals can be identified with natural resistance to an infectious agent, such as BRD or, alternately preferred animals may be identified using a genetic screening test specific to identify the presence of a genotype correlating with resistance to BRD. The animal can then be purchased from that owner and used for subsequent testing or breeding to propagate that trait.

The methods of the present invention contemplate the identification and use of any trait that may be economically important in an animal, particularly in cattle. Merely as exemplification, some desired phenotypic traits include, but are not limited to, disease resistance, such as resistance to Bovine Respiratory Disease (BRD); feed efficiency; reduction in methane emissions; embryonic lethality; tenderness; temperament; polledness; quantity of milk or meat produced; marbling of meat produced; structural and udder soundness; pest resistance; heat tolerance; “fleshing” ability; mothering ability; calving ease; and the like. However as will be appreciated by one of skill in the art, the trait of interest will depend upon a variety of factors including the animal of choice (cattle versus pigs versus poultry and so on); the type of farm, ranch or facility used; methods used to identify the desired trait (for example, phenotypic observation versus SNP or other genetic analyses and identification); methods used for passing the trait to progeny (for example, conventional or enhanced breeding techniques including artificial insemination versus gene editing) and the like. One of ordinary skill in the art will recognize that this list is not meant to be exhaustive. Rather, almost any trait of economic importance can be included in accordance with the system and methods of the present invention provided that the trait can be identified and associated with a genotype such that the trait is heritable.

It is also contemplated by the present invention to encompass the evaluation, identification and selection of a desired trait. In one embodiment contemplated by the present invention, a system and associated methods are provided generally directed to identifying at least one desired trait and producing progeny with a greater likelihood of expressing that trait using conventional breeding techniques; evaluating the resulting progeny to optimize the heritability of the desired phenotypic trait; and selecting the progeny for desired outcomes including determining animals that may be useful for breeding versus those animals that may be sold or selected for meat production. For example, a person practicing the invention may evaluate progeny resulting from a breeding cross to determine those exhibiting the desired trait by physically inspecting the progeny.

Other methods of identification and evaluation related to a desired trait are encompassed within the present invention. Methane is a greenhouse gas with a global warming potential 28 times that of CO₂. Because there is significant interest in reducing enteric methane emissions by ruminants, a number of measurement systems are available to quantify such emissions. Commonly available methods and systems include respiration chambers, the use of a SF₆ tracer gas techniques, and even breath sampling techniques (see, Garnsworthy et al, Comparison of Methods to Measure Methane for Use in Genetic Evaluation of Dairy Cattle, Animals 2019, 9, 837; doi:10.3390/ani9100837, 21 Oct. 2019).

One embodiment of the invention encompasses a computer-assisted method for tracking, for example, the breeding, husbandry conditions, genotypic and related phenotypic data and veterinary histories of livestock animals and generating a profile of the animal or group of animals. This method includes the use of a computer system encompassing a computing device comprising a processor, a data storage system, an input system and an output system, and the steps of generating a profile of an animal by inputting into the computing device through the input device genotype data of the animal, wherein the genotype may be defined by a panel of at least two single nucleotide polymorphisms that predict at least one physical trait of the animal, inputting into the programmed computer through the input system welfare data of the animal, correlating the inputted welfare data with the phenotypic profile of the animal using the processor and the data storage system, and outputting a profile of the animal or group of animals to the output device. Welfare data encompasses, but is not limited to, data such as at least gas emissions data, feed intake data, water intake data, health and behavior data, treatment data, sample data, parentage data, measurement data, personalized animal data, breeding history, a welfare profile, diagnostic data, and quality control data, genomic data, veterinary history clinical and veterinary guidelines, published research data, or combinations thereof. The methods of the invention may be applied to any animal, but most advantageously to a livestock animal such as a dairy or beef bovine, a sheep, a goat, a horse, a pig, a llama, a bird such as a chicken, turkey, duck or quail, and the like. In one embodiment of the invention, the genotype of the animal may be further defined by a panel comprising one SNP predicting a physical trait of the animal. In other embodiments of the invention, the genotype is further defined by a plurality of panels, each panel having at least two SNPs predicting a physical characteristic of the animal. The SNPs may be derived from genes responsible for a physical trait of the animal that may be of interest to the breeder or raiser of the animal(s), such as, but not limited to, any ob, BGHR, calpain, calpastatin, CXCR2. DGAT1, FAA, TIMP2, IGF, IGF-2, POMC, neuropeptide Y, leptin receptor, thyroglobulin, UCP2 and UCP3, or a combination thereof. The methods of the invention further encompass transmitting the profile via telecommunication, telephone, videoconference, or mass communication, to a computer presentation. The methods according to the invention may also encompass the steps of inputting into the programmed computer the desired performance parameters of the livestock animal or population of livestock animals and correlating the required performance parameters of the livestock animal or population of livestock animals to a specific desired performance requirement of a customer.

In an embodiment of the present invention, each individual animal in the testing population is fitted with an electronic identification device (EID). This tag is applied to each animal's ear. The animal is weighed and placed in a pen in which there is a machine that can accurately measure methane and other gases. In one embodiment of the present invention, a C-Lock Greenfeed™ machine is used. GreenFeed™ is a turn-key system designed to measure gas fluxes, or gas emissions data, of Methane (CH₄), Carbon Dioxide (CO₂), and optionally, Oxygen (O₂), and Hydrogen (H₂) from individual animals (Hristov A N, Oh J, Giallongo F, Frederick T, Weeks H, Zimmerman P R, Harper M T, Hristova R A, Zimmerman R S, Branco A F. The Use of an Automated System (GreenFeed) to Monitor Enteric Methane and Carbon Dioxide Emissions from Ruminant Animals). It is also possible to aggregate emissions data from individual animals and determine herd averages. The system is typically configured to offer a small amount of pelleted bait attractant to entice the animals to visit multiple times per day. The gas emissions data is logged then processed allowing the user to easily access a summarized report of calculated fluxes. The GreenFeed™ machine is equipped with an EID tag reader that the EID tag in the animal's ear must pass by in close proximity to record which animal is using the machine at that time. Once that EID is read the GreenFeed™ system then begins to record the emissions from that animal. As different animals activate the system, each individual's unique emission record is digitally saved and is available for analysis. The test is active over a required period of time to collect adequate and accurate gas emissions data on each individual animal. The analysis of these individual records is the method used to select animals with favorable traits and begin the search for genetic selection. Progeny may be produced by any of a variety of reproduction methods including conventional breeding, IVF, cloning, embryo transfer and the like.

In another aspect of the present invention, systems are contemplated and designed to obtain phenotypic information on animal performance using standard as well as novel feed intake data capture systems. Two embodiments of such systems are the C-lock Smartfeed™ system or the Vytelle Sense™ system.

In an embodiment of the present invention, each individual animal in the testing population is fitted with an electronic identification device (EID). This tag is applied to each animal's ear. The animal is weighed and placed in a pen in which there is a C-Lock SmartFeed™ machine. The SmartFeed™ system integrates RFID, load cell and feed bin technology to continuously log data to determine the feed intake per visit per animal, ultimately allowing the user to make informed decisions regarding the feed efficiency of the herd's genetics. Each SmartFeed™ unit transmits data independently, using either a Wi-Fi or cellular network, to a cloud-based server, and is accessible in real-time from a desktop or smart device. In the event a connection is lost, the data will be stored on the unit until the connection is restored or, alternatively, is retrieved with a USB connection. SmartFeed™ can alert the user when specific animals have either low feed consumption, or do not visit after a certain time so that problems with individual animals can be addressed quickly. The feed intake data is logged and then processed, allowing the user to easily access a summarized report of calculated fluxes. The SmartFeed™ machine is equipped with an EID tag reader that the EID tag in the animal's ear must pass by in close proximity to record which animal is using the machine at that time. Once that EID is read the SmartFeed™ system then begins to record the intake of that animal. As different animals activate the system each individual's unique intake record is digitally saved and is available for analysis. The test is active over a required period of time to collect adequate and accurate data on each individual animal. The analysis of these individual records is the method used to select animals with favorable traits and begin the search for genetic selection

In another aspect of the present invention, systems are contemplated and designed to obtain phenotypic information on animal performance using standard as well as novel water intake data capture systems. One embodiment of the present invention is the commercially available system known as the C-lock SmartWater™ system (Sarah Reilly, et al., 418 Evaluation of an automated system for measuring individual animal water consumption, Journal of Animal Science, Volume 102, Issue Supplement_3, September 2024, Page 4).

In a further embodiment of the present invention, each individual animal in the testing population is fitted with an electronic identification device. This tag is applied to each animal's ear. The animal is weighed and placed in a pen in which there is a C-Lock SmartWater™ machine. The SmartWater™ System allows the user to measure water intake. The system is self-contained, wireless, and portable for individual animals. The simple design makes collecting individual water intake data accurate, affordable, and easy. The tank holds 30 gallons of water measuring (4 ft3/0.1 m3) and has a built-in heater to operate in cold conditions. The SmartWater™ System has two options for connecting water. The first is exposed on the side of the base. The second option is to have conduit running up and into the base. SmartWater™ can alert the user when specific animals have either low water consumption, or do not visit after a certain time so that problems with individual animals can be addressed quickly. The water intake data is logged then processed, allowing the user to easily access a summarized report of calculated fluxes. The SmartWater™ machine is equipped with an EID tag reader that the EID tag in the animal's ear must pass by in close proximity to record which animal is using the machine at that time. Once that EID is read the SmartWater™ system then begins to record the intake of that animal. As different animals activate the system each individual's unique intake record is digitally saved and is available for analysis. The test is active over a required period of time to collect adequate and accurate data on each individual animal. The analysis of these individual records is the method used to select animals with favorable traits and begin the search for genetic selection.

In another aspect of the present invention, systems are contemplated and designed to obtain phenotypic information on animal health and behavior, called health and behavior data herein, including resistance or susceptibility to Bovine Respiratory Disease and other common pathogens using standard as well as novel health data capture systems such as the Merck Animal Health SenseHub™ ear tag system. Such a system is useful for monitoring fever in an animal, including in cattle, providing an objective assessment of a clinical sign that can be used to assess progeny to produce, for example, an Expected Progeny Difference (Expected Progeny Differences Trait Definitions and Utilizing Percentile Tables. Sean Bessin and Darrh Bullock, Animal and Food Sciences, ASC-211: Expected Progeny Differences: Trait Definitions and Utilizing Percentile Tables (uky.edu)) or Predicted Transmitting Ability or other similar recognized measure of genetic merit.

In a further embodiment of the present invention, each individual animal in the test population is fitted with a tag equipped with technology that can monitor, analyze and transmit that animal's biological parameters, or health and behavior data, such as the SenseHub™ ear tag. These parameters include body temperature and physical movement. When there is a higher body temperature or lack of movement detected by the monitoring tag, that animal is checked by professional caretakers. If the caretakers determine that the animal needs care, the animal is removed from the test population and further evaluated then treated if necessary, generating treatment data. The caretakers record the reason the animal is treated. The test is active over a required period of time to collect adequate and accurate treatment data on each individual animal. The analyzation of these individual records is the method used to select animals with favorable traits for heath and resistance to disease such as Bovine Respiratory Disease (BRD) and begin the search for genetic selection

In another aspect of the present invention, systems are contemplated and designed to obtain phenotypic information on animal performance using standard as well as novel weight and weight gain data capture systems such as the C-lock SmartScale™ system or the Vytelle Sense™ system.

In a further embodiment of the invention, each individual animal in the testing population is fitted with an electronic identification device. This tag is applied to each animal's ear. The animal is weighed and placed in a pen in which there is a C-Lock SmartScale™ machine or Vytelle Sense™ machine. SmartScale is a wireless scale system that captures animal weight, performance, and behavior each time it drinks water. This data is collected during a few days before the animal is harvested. At harvest time the actual live weight recorded by the SmartScale™ machine is analyzed along with the harvested hot carcass weight to determine the individual animal's dressing percentage or yield. The test is active over a required period of time to collect adequate and accurate weight data on each individual animal. The analysis of these individual records is the method used to select animals with favorable traits and begin the search for genetic selection.

Contemplated in this invention is an assessment of combinations of genetic merit for various synergistic or antagonistic traits of economic significance, to derive an index or weighted average of the genetic or economic merit of the animals, such as the Net Merit Index in dairy cattle, a tool used in dairy cattle breeding to assess the lifetime profitability of an animal by considering both production and functional traits. The genetic contribution of several traits, like milk production, conformation, health and fitness, are factored into Net Merit. Traits are weighted based on their genetic impact on dairy farm profitability. Net Merit was developed in 1994 by renowned USDA geneticists and is updated periodically with new traits and the latest research and economic values. Since 2021, Net Merit has included 39 individual traits for Holsteins. Not all traits are calculated for all breeds, so the Net Merit formula differs slightly between breeds.

The present invention also contemplates a system to capture biologic material, including DNA and RNA, for systematic evaluation through genotyping or sequencing. This would include individual animal identification and association of that animal ID with a corresponding sample ID. One of ordinary skill will recognize any number of common systems will work to effectively link a sample from a test animal to that animal. Commercial systems are also available such as used in the Allflex Tissue Sampling Unit (College Station, TX) which offers a method and system to collect samples for genetic testing and can pair the tissue sample unit with an Electronic ID and/or visual tag to link the sample with the animal from which the sample was derived. The sample collected may be any type of biologic material from the animal including but not limited to hair samples with root bulb, semen, meat or other bodily tissue, saliva or oral swab including cheek swab, oocytes, cells extracted from a developing blastocyst, and so on. The sample ID, including bar code or other identifying feature, and corresponding animal ID are then entered into a data system. In a further embodiment, the tissue sample is subjected genotyping or sequencing, including low pass sequencing and imputation, and the resulting genomic data is associated with the animal identification and sample identification and stored in the Data System.

In another aspect of the present invention, systems are contemplated for collection of biologic material from all animals in the production system at an early age, including but not limited to cells from developing blastocysts (Oliveira C S, et al., Embryo biopsies for genomic selection in tropical dairy cattle. Anim Reprod. 2023 Jul. 24; 20(2):e20230064). Using single nucleotide polymorphism (SNP) genotyping as well as other well-known sequencing technologies, SNPs and other genetic variants are identified and analyzed for their association to superior and/or detrimental production traits. In particular, a sample of genomic DNA from an animal may be evaluated by reference to one or more controls to determine if a SNP, or group of SNPs, in a gene is present. Any method for determining genotype can be used for determining the genotype in the present invention. Such methods include, but are not limited to, amplimer sequencing, DNA sequencing, fluorescence spectroscopy, fluorescence resonance energy transfer (or “FRET”)-based hybridization analysis, high throughput screening, mass spectroscopy, microsatellite analysis, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen and Sullivan, Pharmacogenomics J 2003; 3(2):77-96, the disclosures of which are incorporated by reference in their entireties. One aspect contemplated to be within the present invention is the use of standardized genotyping arrays such as, but not limited to the Illumina Bovine SNP 50™. Other sequencing techniques are well known to those of skill in the art and considered to be within the scope of the present invention. For example, the use of various sequencing techniques (short and long read) at varying levels of coverage of the genome to derive a more complete genome assembly of the animals is contemplated by the present disclosure. A further contemplated use is sequencing at low coverage, or low pass sequencing, following by imputation to full sequence using analytic methodologies known to those of skill in the art (Li J H, Mazur C A, Berisa T, Pickrell J K). The sequencing can also be done by any commercial company, including but not limited to NeoGen. Low-pass sequencing increases the power of GWAS and decreases measurement error of polygenic risk scores compared to genotyping arrays (Genome Res. 2021 April; 31(4):529-537).

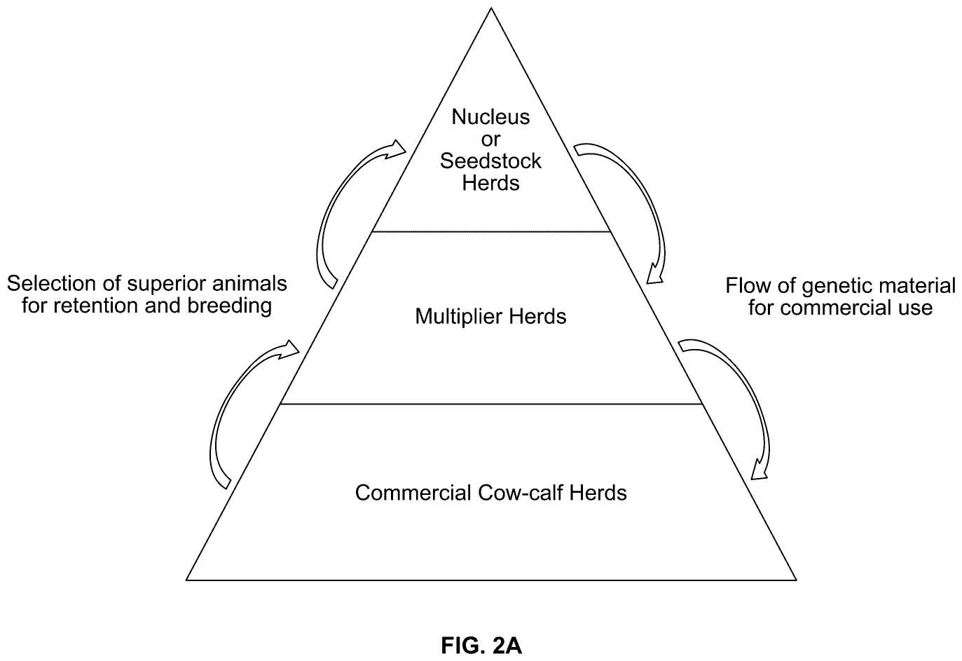

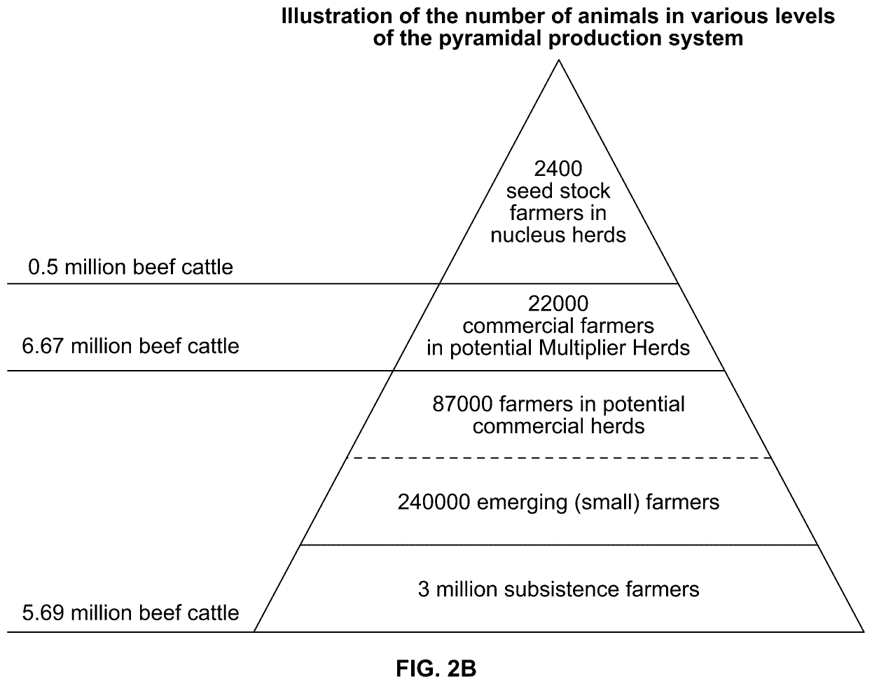

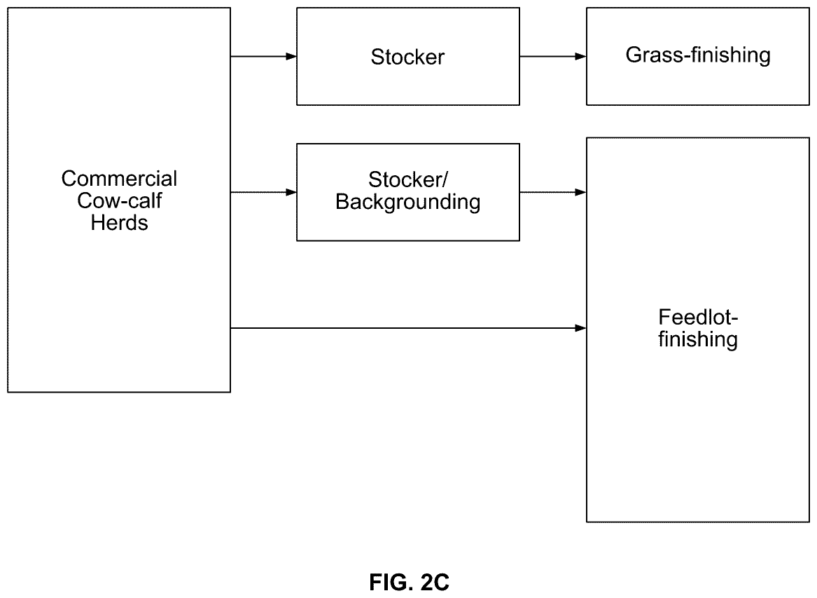

In a further embodiment, the animal ID and sample ID are associated with the individual animal phenotypic measures as described earlier and stored in the Data System. Techniques such as Genome Wide Association Study are then used to examine association between genomic polymorphisms and trait variations. Further those associations are then used to identify superior and detrimental trait variations and the resulting trait associations are used to refine selection of superior breeding animals in future selection decisions in the pyramidal production system as illustrated in FIG. 2 showing the gene flow system that is useful in both beef and dairy cattle production.

Other methods for evaluating a desired trait contemplated to be within the scope of the present invention include techniques to analyze a genotype associated with a desired trait, whether in the potential breeding stock or in the progeny. For example, one such method contemplates the use of single nucleotide polymorphisms (SNP) which refers to a polynucleotide that differs from another polynucleotide by a single nucleotide exchange. It is contemplated that identification of a desired trait can be accomplished by using breed-specific single nucleotide polymorphisms (SNPs) and their haplotypes, in the genome of interest such as a bovine genome, in or near gene(s) encoding polypeptides associated with the trait of interest. For example, a dominant mutation responsible for the polled phenotype and a number of other traits have been identified having a close proximity to a SNP (For example, US published Patent Application No. 2008/0160523A1 to Woodward and Nkrumah, published 3 Jul. 2008; U.S. Pat. No. 7,897,749 to Khatib, granted 1 Mar. 2011; U.S. Pat. No. 7,947,444 to Moore, granted 24 May 2011; and U.S. Pat. No. 8,105,776 to Gill et al, granted 31 Jan. 2012 amongst others). Once a SNP of interest has been identified and breeding stock evaluated for that SNP, conventional and enhanced breeding and selection techniques can be used to produce progeny having the desired trait.

The present invention also contemplates introducing a desired trait into an animal, which in one embodiment is a bovine, using gene editing techniques. CRISPR has been used to generate super-muscular production animals including cattle, pigs, sheep, rabbits and goats (see Jessica Hamzelou, How CRISPR is Making Farmed Animals Bigger, Stronger, and Healthier, MIT Technology Review, 20 Jan. 2023). Scientists have also already used gene editing to produce genetically-altered pigs able to express the Δ12 fatty acid desaturase gene, from spinach, to exhibit higher levels of omega-3 fatty acids in the meat (Saeki et al, Functional Expression of a Δ12 Fatty Acid desaturase Gene from Spinach in Transgenic Pigs, PNAS 101(17): 6361-6366, 5 Apr. 2004). Briefly, following collection of zygotes from super ovulated gilts (≈100 kg of live weight) obtained from a cross between Duroc sows and Landrace×Large White boars. DNA was injected into the male pronuclei of zygotes. Injected embryos were transferred into the oviducts of synchronized recipients or the donor pigs. At birth, a tail biopsy of each piglet was performed and the transgene was examined by Southern blotting. After puberty, transgenic pigs with an active transgene were mated with wild-type pigs and Southern blotting analysis done on the live piglets showed that (38%) cared the transgene.

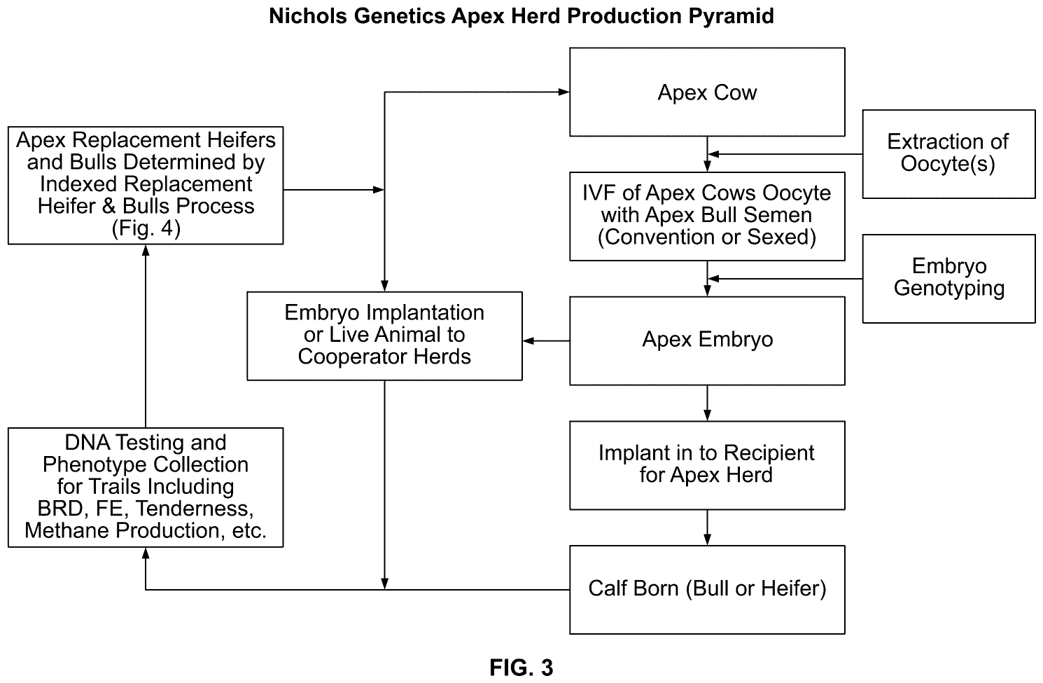

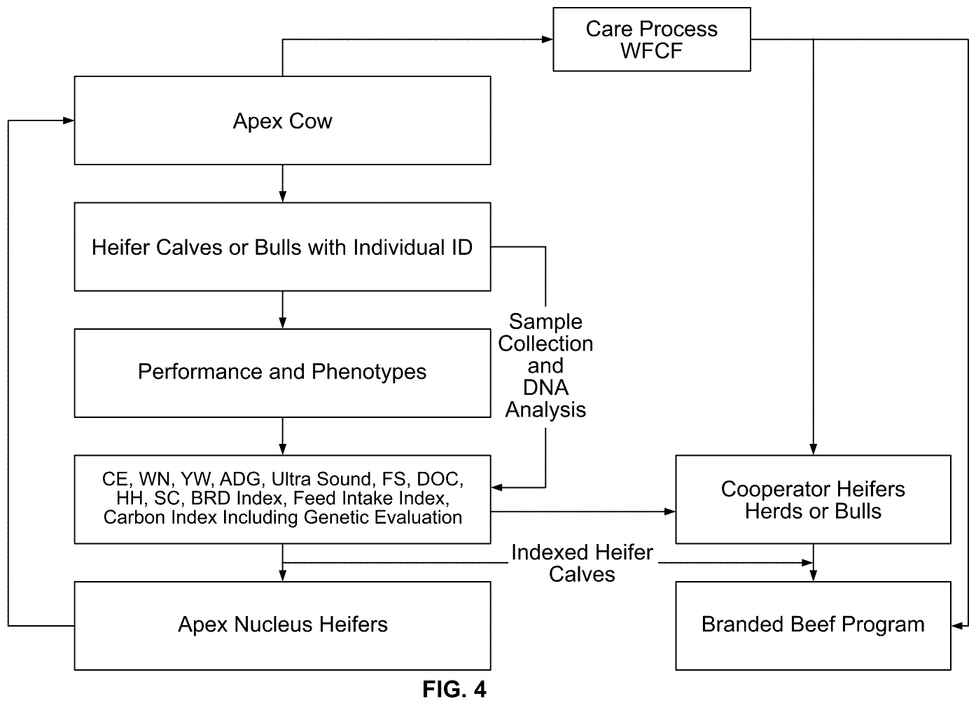

FIG. 3 illustrates the pyramidal scheme that produces replacement heifers and bulls to repopulate the Apex Herd, and to move a desired trait into the second tier of Cooperator Herds that will multiply a trait or even a collection of traits to provide superior genetics in a replacement heifer or a bull, for example. In this illustration, an Apex Cow 1 which demonstrates a superior genetic trait or collection of traits is subjected to enhanced reproductive technologies such as super-ovulation and oocyte collection. Using in-vitro fertilization (IVF), for example, the embryo(s) are fertilized with conventional or sexed semen to produce a viable blastocyst 3 which may be genotyped using cells extracted from the developing blastocyst. Whether genotyped or not, the embryo is subsequently implanted into a suitable recipient female 4 for the Apex Herd or may be implanted into a suitable recipient female for a Cooperator or Multiplier Herd 7. When the calf is born-whether a male or female and whether in the Apex Herd or a Cooperator herd, the resulting progeny has a DNA sample taken and matched with its unique animal ID. That sample is then genotyped 6 and the animal begins the process of phenotype collection for all available traits including but not limited to BRD resistance, feed intake and feed efficiency, tenderness, methane production, and the like. The animal may then be formally evaluated as in an Apex Replacement Heifer and Bull selection process 8 as further defined in FIG. 4. Animals that meet the threshold for superior genetic merit are returned to the Apex Herd as replacements for Apex Cows 1 or may move to Cooperator Herds 7 for use in multiplying superior genetics.

FIG. 4 is a schematic which provides an outline of the DNA and phenotype collection process in an embodiment according to the present invention. Progeny, male or female 2, from Apex cows 1 are produced from enhanced reproductive techniques or from conventional breeding. At birth, the animal is individually identified and a sample taken for DNA analysis and storage. Using an accepted genotype tool known to those of skill in the art, including but not limited to fixed array genotyping, such as the Illumina® Bovine SNP 50 panel or sequencing, a genomic analysis is provided. The animal then enters a rigorous process for phenotype collection using a system such as C-Lock SmartFeed® or Merck SenseHub® or the like 3. Any number of other analyses or diagnostic tests may be performed including, but not limited ultrasound analysis, conformation determination, and a genetic evaluation 4. Depending upon the genetic merit of the individual animal, it is selected for return to the Apex Herd 5 or it is directed to the cooperator or multiplier herds 6 from which the resulting progeny are used in commercial herds to produce animals for a branded beef program 7 such as the Nichols Genetics Index. All of the animal production, whether directed to the Apex or Cooperator herds, or in commercial production herds, is carried out under the direction and auspices of the Care Process or similar certification and traceability program 8.

The present invention further relates to methods and systems, including network-based processes, to manage the data relating to specific animals and herds of animals; veterinarian care; diagnostic and quality control data; breeding and management of livestock with predictable traits; husbandry conditions; food safety information; quality control audit information; safety information; collection of data from remote locations including field locations; and the like.

Computer Readable Programming: Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. Thus, it is impossible for any program to damage the execution of any of the other programs running on the system. However, the programs have no way to exchange any information except through the operating system (or by reading files stored on the file system).

Multi-process computing is similar to multi-tasking computing, as the terms task and process are often used interchangeably, although some operating systems make a distinction between the two. The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

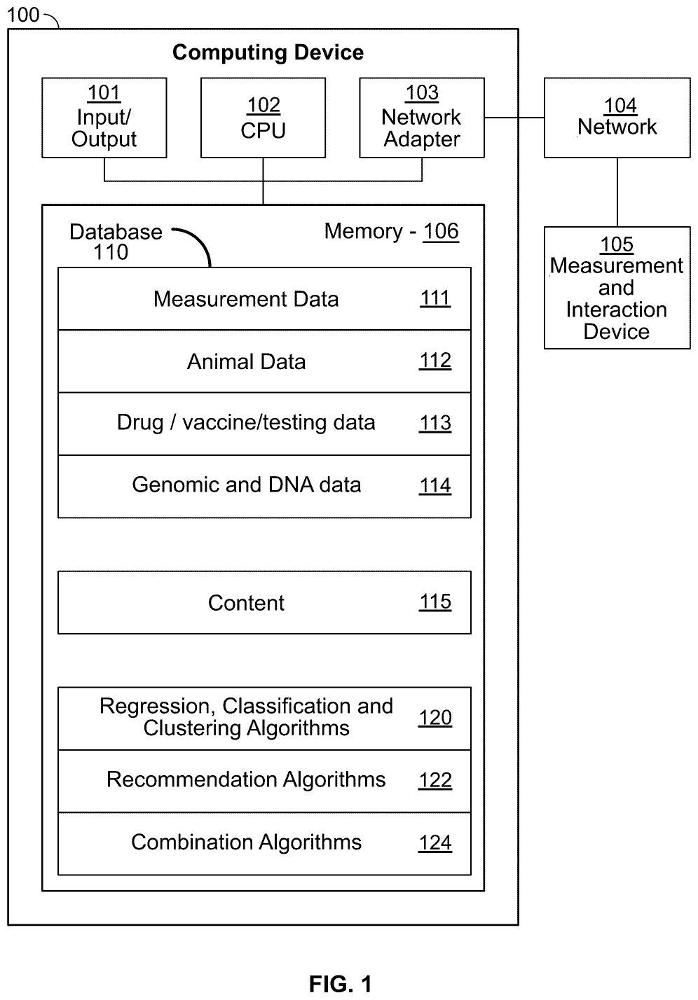

An example of a system is illustrated in FIG. 1. A computing device 100 is depicted along with a processing unit 102 (e.g. a central processing unit (CPU), but also encompassing graphics processing units (GPUs) or even multiple processors or cores), an input/output device 101, a network adapter 103, memory 106, and database 110. The network adapter 103 connects the computing device 100 to a network 104 which may include a measurement and interaction device 105. Various data, programs, and algorithms may be stored on the memory 106. Within the database 110 of the computing device 100 reside data including, but not limited to personalized animal data 112 including results of physical exams, phenotypic observations or genomic results including blood panels, histology/pathology results; endoscopy data including images, videos, stool/microbiome testing, testing of breath/air including measuring methane and carbon dioxide levels expelled, as well as results of imaging including X-Rays, MRI, CAT scan, fluorescence and the like. Other data contemplated to reside within the database 110 of the computing device 100 include feed/drug/vaccine data 113 comprising information about types and amounts of feed, drugs such as antibiotics given, and additives provided to the animal. It is also contemplated that database 110 of the computing device 100 will comprise measurement data 111 including, but not limited to information from scales, sensors, ear tags, and the like are encompassed within the present invention and may be collected. In another embodiment, data, such as electronic health records and genetic testing may be collected and analyzed. In addition to measurement data 111, individual animal data including parentage data 112, feed/drug/vaccine data 113, genomic and DNA data including SNP data 114, additional data may be gathered and stored as desired by the user. Content, including but not limited to, a database of genetic testing, therapeutic treatments, recorded audio/video, written material and the like is gathered and stored in 115. Some data may reside in other locations connected to the network including, but not limited to a database of clinical or veterinary guidelines; a database of published research and the like.

Various programs, sub-routines or algorithms such as classification algorithms 120, recommendation algorithms 122, analysis and comparison algorithms 124, machine learning algorithms (ML), artificial intelligence (AI) algorithms, and combinations thereof may reside on the memory 106 of the computing device 100. ML algorithms are a part of artificial intelligence and use an assortment of accurate, probabilistic, and upgraded techniques that empower computers to pick up from the past point of reference and perceive hard-to-perceive patterns from massive, noisy, or complex datasets. AI algorithms are a set of instructions or rules that enable machines to learn, analyze data and make decisions based on that knowledge. These algorithms can perform tasks that would typically require human intelligence, such as recognizing patterns, understanding natural language, problem-solving and decision-making.

Database 110 of the present invention may also allow for the storage and retrieval of data collected from a number of sources including from clients; production facilities; research institutions, ranches, feedlots and the like. Traditional file systems and filers have their strengths, and high-performance file sharing needs still exist within data centers, so existing filers and file systems fulfill that need. Cloud storage, on the other hand, with associated network latencies is not always a good fit for certain use cases. But cloud storage excels with Internet applications where the generation of content can be viral and where it can be virtually impossible to predict capacity or access needs. Cloud storage is also ideal in the case of Web 2.0 applications which promote collaboration between hundreds and thousands of users sharing the same files or objects.

Systems and methods are well known in the art for performing data storage operations, including content indexing, containerized deduplication, and policy-driven storage, within a cloud environment. The systems support a variety of clients and storage devices that connect to the system including using a cloud environment, which permits data transfer over wide area networks, such as the Internet. Such systems may also allow available storage devices to include cloud storage sites. Methods for content indexing data stored within a cloud environment to facilitate later searching, including collaborative searching are also known. For example, see U.S. Pat. No. 11,907,168 to Prahlad et al; U.S. Pat. No. 11,934,540 to Ortiz et al; US Published Application 2022/030903 to Gutierrez and Faulkner; and the like (the contents of which are incorporated in their entirety). The present system also contemplates methods for identifying suitable storage locations, including suitable cloud storage sites, for data files subject to a storage policy.

A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire. Computer readable program instructions described herein can be downloaded to respective computing/processing devices from a computer readable storage medium or to an external computer or external storage device via a network 104, for example, the Internet, a local area network, a wide area network and/or a wireless network. The network 104 may comprise copper transmission cables, optical transmission fibers, wireless transmission, routers, firewalls, switches, gateway computers, and/or edge servers. A network adapter card or network interface 103 in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. The flowchart and block diagrams as in FIG. 1 illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s).

In alternative embodiments, the functions noted in the blocks may occur out of the order noted in the Figure. For example, two blocks shown in succession may, in fact, be executed substantially concurrently, or in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or that carry out combinations of special purpose hardware and computer instructions. Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

The management system of the present invention further includes but is not limited to i) a means for the collection of data, including data from individual animals as well as herds. The data can be gathered and collected actively from, for example, observation of an animal or passively from connected technology such as an instrument to weigh an animal or input from a laboratory such as analysis of a DNA sample; ii) a means of storing the collected data in a data repository; iii) a means for generation of an individualized database from the data collected and stored at each step or phase of the method of the present invention; iv) a means for analysis of data in the individualized data base using at least one algorithm; and v) a means of communication of information during one or more stages of the present method including, but not limited to, data input and data output; statistics generated from the collected data; use of the data to aid in the evaluation and selection of a particular animal to optimize a trait in the resulting population; and the like.

Data analysis as contemplated by the instant invention includes generating and using machine learning algorithms and providing individual level data analysis; population level data analysis; and additional analysis including clustering by similar characteristics such as a desired trait, clinical symptoms and/or signs, and the like. A computing device may execute one or more artificial intelligence and/or machine learning software programs to generate and update the algorithms, generate and update health scores of individual animals and herds, identify trends among herds, or other similar analysis. One of ordinary skill in the art will recognize that the artificial intelligence and machine learning software may execute various artificial intelligence and machine learning algorithms and processes, such as generalized linear models, random forests, support vector machines, unsupervised and/or supervised clustering, and deep learning (e.g., neural networks), among others. In some examples, machine learning software may “learn” (e.g., update data processing algorithms according to historical data trends) from training data, which, in some instances, includes labeled data. The machine learning software may implement a feedback loop, in which feedback may be presented based on at least the genotypic and phenotypic profile of the progeny.

Statistical analysis is completed according to desired outputs including baseline genetic information related to a desired trait or traits; general health parameters; physical parameters such as measurements of phenotypic traits; and the like, with additional outputs generated that are useful in a method in accordance with the present invention. For example, one embodiment of the invention contemplates calculating and predicting animals which gain more weight than their contemporaries while eating the same amount of food, thus identifying and predicting an animal which is likely to carry a trait resulting in feed efficiency. In this embodiment of the invention, a model predicting an animal that is feed efficient applied by an operations server or other computing device of the digital service may be trained and re-trained using any number of known statistical and regression algorithms against a predetermined set of data fields. In some cases of this embodiment, a random forest regression or other similar machine learning or artificial intelligence model can be trained to predict outcomes utilizing database records for individual animals who express a phenotypic trait of interest. In an automated implementation of this embodiment, the operations server may retrieve values of certain data fields from user records that are stored in a database and may then reapply the particular training algorithm.

Other outputs of onboarding data collection and analysis include, but are not limited to, the recommendation of personalized disease management programs including at least one desired trait such as resistance to a disease such as Bovine Respiratory Disease (BRD). The use of the management tool to identify an animal which may carry this trait may include a management system to monitor temperatures in a herd using, for example, an ear tag which can transmit data including body temperature and the like at various intervals to identify an animal which may be infected with a virus such as BRD, and conversely identify animals who even in the midst of a herd exposure to this virus appear to be resistant to infection. Such an electronic ear tag to track biometric data to monitor the health of each animal is well known in the art to determine and collect measurements such as temperature using, for example, an infrared thermistor or track animal activity using a built-in accelerometer (see, for example, Merck's SenseHub™ ear tag, Medisim's TAGim™, and Fever Tag).

The use of the management tool to identify an animal which may carry this trait may include a management system to monitor feed intake to assess feed efficiency (FE) using, for example, and individual animal feed intake monitoring systems such as the C-Lock™ feed intake and methane emission monitoring system. Such a system to track biometric data to monitor the feed intake and methane production of each animal is well known in the art to determine and collect measurements for efficiency and environmental impact.

Standard references may be consulted to determine the type of analysis methodology to use for analyzing the data collected by a management system according to the present invention including, for example, Introduction to Statistical Analysis (1969), Dixon and Massey; McGraw-Hill Publishing, New York and Handbook of Data Analysis (2004), Hardy and Bryman, SAGE Publications (the contents of which are incorporated by reference in their entirety). In another example of a standard methodology, the type of analysis methodology includes assessment of the pedigree or relatedness of an animal to its contemporaries and parents, and calculation of its relative genetic merit through estimation of the Expected Progeny Difference (EPD) or Predicted Transmitting Ability (PTA).

A system and method in accordance with the present invention is useful for optimizing the selection of production animals to aid in the identification and evaluation of a desired trait. In one embodiment the identification and evaluation can identify a desired trait based upon phenotype, but it is also contemplated that identification of a trait can be accomplished by other means including genetic assays such as SNP analysis. The trait can then be introduced into progeny using classical breeding techniques, Artificial insemination, or gene editing.

As a mean of illustration, one embodiment for a method according to the present invention useful for producing a bull with a desired trait or traits is shown in the following Table:

TABLE 1
Process for producing a bull with a desired trait

| Stage | Program | Testing (exemplary) |
|---|---|---|
| Gestation | 3rd party certification | |
| Cow/calf | 3rd party certification | |
| Wean calf | 3rd party certification | DNA test, optional test for |
| | | phenotype, parentage and |
| | | trait profile |
| Develop bull | | CI test, FE test, |
| or replacement | | ultrasound, performance, |
| heifer | | phenotype, EPD, NFF index |

In addition to including standard animal husbandry practices, the management program for gestation and the cow/calf stage is directed to the cow during pregnancy and, subsequently, to the cow/calf pair before weaning. This stage includes at least one optional certification program which, for example, may be used to provide transparency and traceability in beef production. One such program, provided only for exemplary purposes, is the CARE program from IMI Global which provides a branded program for producers who meet designated standards. Earning the brand provides a consumer assurance that the producer has raised the animal in accordance with certain attributes that cannot be verified by simply looking at the product. Generally, the three standards of the CARE program are directed to 1) Animal Care to reinforce animal care measures that are being taken in cattle production including in nutrition, environment, health, and behavioral interactions; 2) Environmental Stewardship to support the maintenance of a sustainable environment by promoting practices that encourage waste reduction, management of natural and renewable resources and reducing our carbon footprint; and 3) People & Community to reinforce efforts to ensure workers are appropriately trained, working in a safe environment and fairly compensated.

The stage directed to the weaned calf still may encompass a third-party certification program, but also will include testing that will aid in the identification and evaluation of the desired trait amongst other purposes. For example, one of more samples including blood and other fluids, ear punch samples, and samples comprising tissue, hair and the like may be obtained for DNA testing and analyzed for a variety of reasons including but not limited to parentage verification, genomic profiling, and genetic health. Parentage verification will help determine which bulls sire the best calves as well as identify infertile bulls. Genomic profiling may help a producer identify carriers of a gene that is not desirable. By breeding a bull carrying a defective gene to a non-carrier, a producer may be able to breed that defect out of the herd. Genomic profiling is useful for identifying desired traits from new bulls or semen but may also be used to select replacement heifers and for selecting animals for certified beef product production. The final stage in the example provided above is to develop a bull or replacement heifer which can be for a variety of end uses including a certified beef product. During this stage, a number of measurements and other tests may be performed. In addition to phenotypic or genomic observation, some examples of testing include, but are not limited to body weight measurement, conformation and/or frame score, birth, weaning and yearling weight, feet score, performance tests including general disposition, average daily gain, carbon emission (CI) test, feed efficiency (FE) test, ultrasound testing for marbling and backfat or the like, or other phenotype measurements, calculation of expected progeny difference (EPD) or other objective and calculated measure of genetic merit, or evaluation of any combination of traits to create a composite index of the genetic merit of the animal such as the Nichols Family Farm (NFF) index, or $Beef as described by the American Angus Association and the like. Bulls or replacement heifers with a particular trait or background may be used for breeding purposes or for semen and or for production of oocytes for in-vitro fertilization or the like and may be sold to a cooperative or other farm or ranch to propagate that genetic background. Bulls or replacement females which may not be as desirable may be used as steers or feeder heifers and eventually will be sold into a market as a certified beef product. In one embodiment, the present invention contemplates labelling and selling the meat from such a steer or heifer as a branded product because the identification and heritability of a genetic trait, such as tenderness and the certification of the production process such as the Canadian Roundtable on Sustainable Beef, can provide a guarantee to the consumer buying the end-product as to the sustainability and wholesomeness.

From the above description, it can be seen that the present invention provides a system, computer program product, and method for the efficient execution of the described methods and techniques. References in the claims to an element in the singular is not intended to mean “one and only” unless explicitly so stated, but rather “one or more.” All structural and functional equivalents to the elements of the above-described exemplary embodiment that are currently known or later come to be known to those of ordinary skill in the art are intended to be encompassed by the present claims. No claim element herein is to be construed under the provisions of 35 U.S.C. section 112, sixth paragraph, unless the element is expressly recited using the phrase “means for” or “step for.”

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art. It is to be understood that the particular examples, materials, amounts, and procedures are to be interpreted broadly in accordance with the scope and spirit of the invention as set forth herein.

### EXAMPLES

### Example 1: Measurement of Methane Emissions

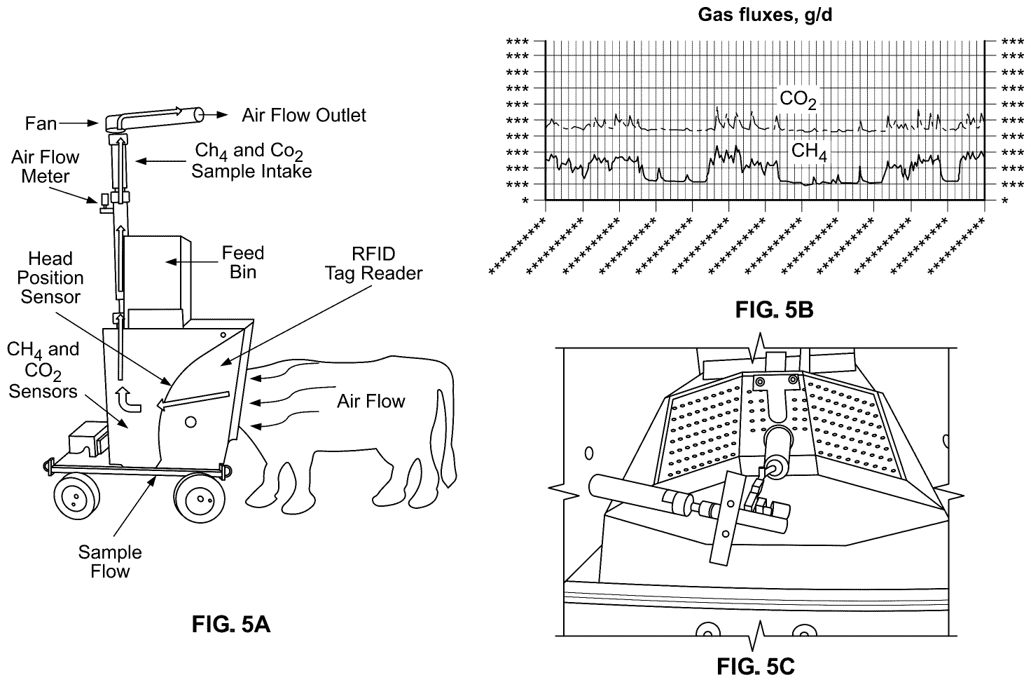

Each individual animal in the testing population is fitted with an electronic identification device. This tag is applied to each animal's ear. The animal is weighed and placed in a pen in which there is a C-Lock Greenfeed™ machine. GreenFeed™ is a turn-key system designed to measure gas fluxes of Methane (CH₄), Carbon Dioxide (CO₂), and optionally, Oxygen (O₂), and Hydrogen (H₂) from individual animals. It is also possible to aggregate emissions data from individual animals and determine herd averages. The system is typically configured to offer a small amount of pelleted bait attractant to entice the animals to visit multiple times per day (FIGS. 5A-5C). The gas emissions data is logged then processed allowing the user to easily access a summarized report of calculated fluxes (FIG. 5B). The GreenFeed™ machine is equipped with an EID tag reader that the EID tag in the animal's ear must pass by in close proximity to record which animal is using the machine at that time (FIG. 5A). Once that EID is read the GreenFeed™ system then begins to record the emissions from that animal. As different animals activate the system each individual's unique emission record is digitally saved and is available for analysis. The test is active over a required period of time to collect adequate and accurate data on each individual animal. The analyzation of these individual records is the method used to select animals with favorable traits and begin the search for genetic selection.

### Example 2: Measurement of Feed Intake, Feed Efficiency and Residual Feed Intake

Each individual animal in the testing population is fitted with an electronic identification device (EID). This tag is applied to each animal's ear. The animal is weighed and placed in a pen in which there is a C-Lock SmartFeed™ machine or a Vytelle Sense™ feed intake machine. The SmartFeed™ system integrates RFID, load cell and feed bin technology to continuously log data to determine the feed intake per visit per animal, ultimately allowing informed decision-making regarding, for example, the feed efficiency of a herd's genetics. Each SmartFeed™ unit transmits data independently, using either a Wi-Fi or cellular network, to a cloud-based server, and is accessible in real-time from a desktop or smart device. In the event a connection is lost, the data will be stored on the unit until the connection is re-stored or alternatively is retrieved with a USB connection. SmartFeed™ can alert the user when specific animals have either low feed consumption, or do not visit after a certain time so that problems with individual animals can be addressed quickly. The feed intake data is logged then processed, allowing the user to easily access a summarized report of calculated fluxes. The SmartFeed™ machine is equipped with an EID tag reader that the EID tag in the animal's ear must pass by in close proximity to record which animal is using the machine at that time. Once that EID is read the SmartFeed™ system then begins to record the intake of that animal. As different animals activate the system each individual's unique intake record is digitally saved and is available for analysis. The test is active over a required period of time to collect adequate and accurate data on each individual animal. The analyzation of these individual records is the method used to select animals with favorable traits and begin the search for genetic selection.

### Example 3: Measurement of Water Intake

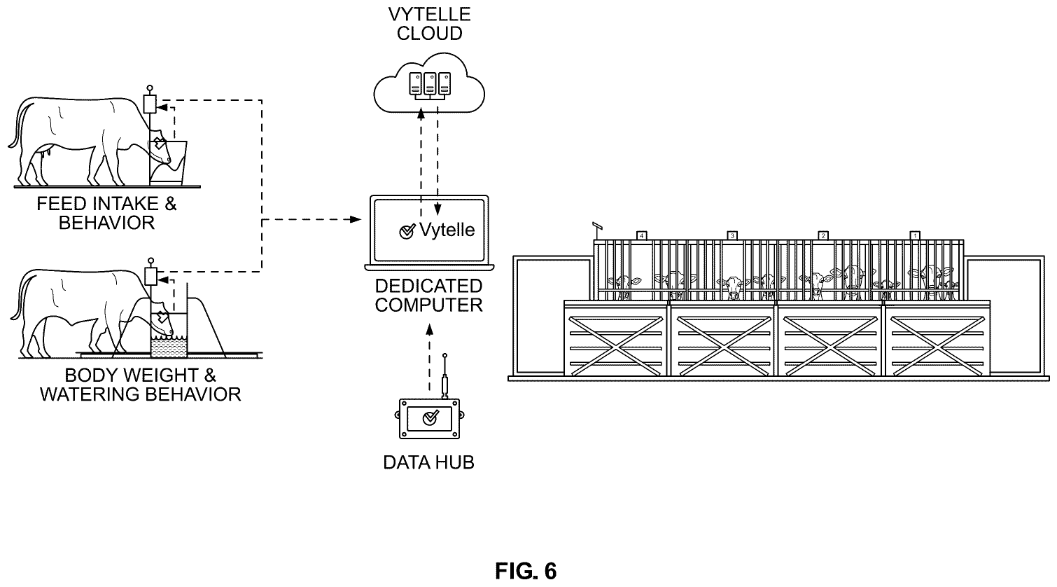

FIG. 6 illustrates a system to measure water intake. Each individual animal in the testing population is fitted with an electronic identification device (EID) applied to each animal's ear. The animal is weighed and placed in a pen in which water consumption can be measures. Commercial machines are available to measure water consumption and store the data including the C-Lock SmartWater™ machine. The tank holds 30 gallons of water and water is delivered to an individual animal and consumption measured. SmartWater can alert the user when specific animals have either low water consumption, or do not visit after a certain time so that problems with individual animals can be addressed quickly. The water intake data is logged then processed, allowing the user to easily access a summarized report of calculated fluxes. The SmartWater™ machine is equipped with an EID tag reader that the EID tag in the animal's ear must pass by in close proximity to record which animal is using the machine at that time. Once that EID is read the SmartWater system then begins to record the intake of that animal. As different animals activate the system, each individual's unique intake record is digitally saved and is available for analysis. The test is active over a required period of time to collect adequate and accurate data on each individual animal. The analyzation of these individual records is the method used to select animals with favorable traits and begin the search for genetic selection.

### Example 4: Measurement of Body Temperature, Movement and Health Parameters

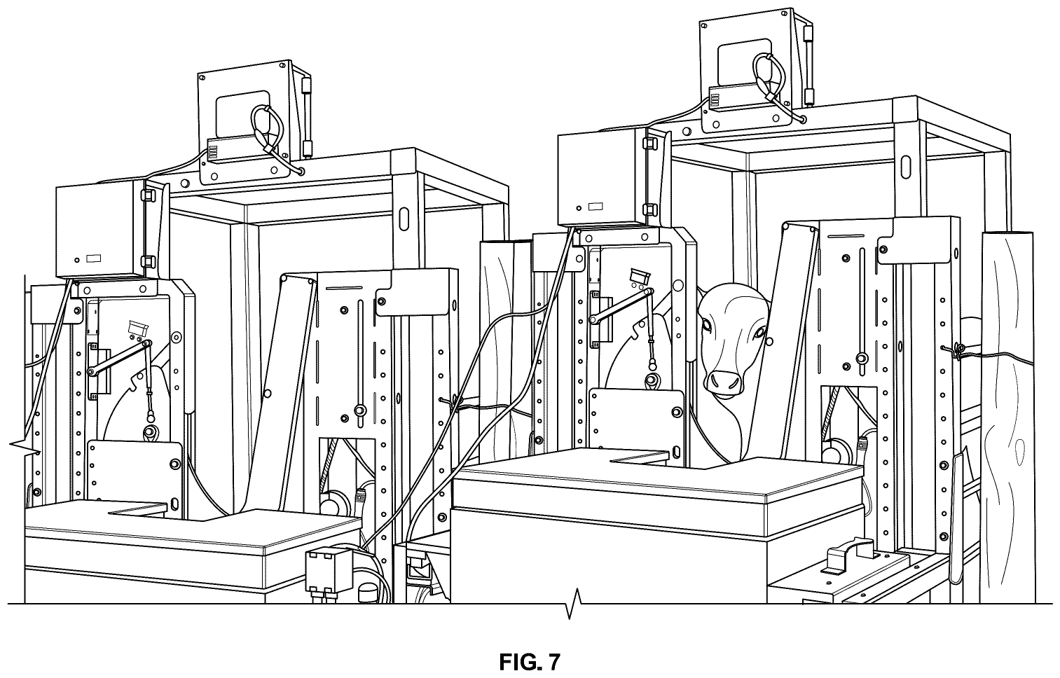

FIG. 7 is illustrative of a system where each individual animal in the test population is fitted with an ear tag equipped with technology that can monitor, analyze and transmit that animal's biological parameters. One such commercially-available tag is the SenseHub™ ear tag. The parameters measured and recorded include body temperature and physical movement. When there is a higher body temperature or lack of movement detected by the monitoring tag, that animal is checked by professional caretakers. If the caretakers determine that the animal needs care, the animal can be removed from the test population and further evaluated and treated if necessary. The test is active over a required period of time to collect adequate and accurate data on each individual animal. The analysis of the individual records can be used to identify animals with favorable traits and select such animals for further purpose such as conventional breeding.

### Example 5: Measurement of Weight

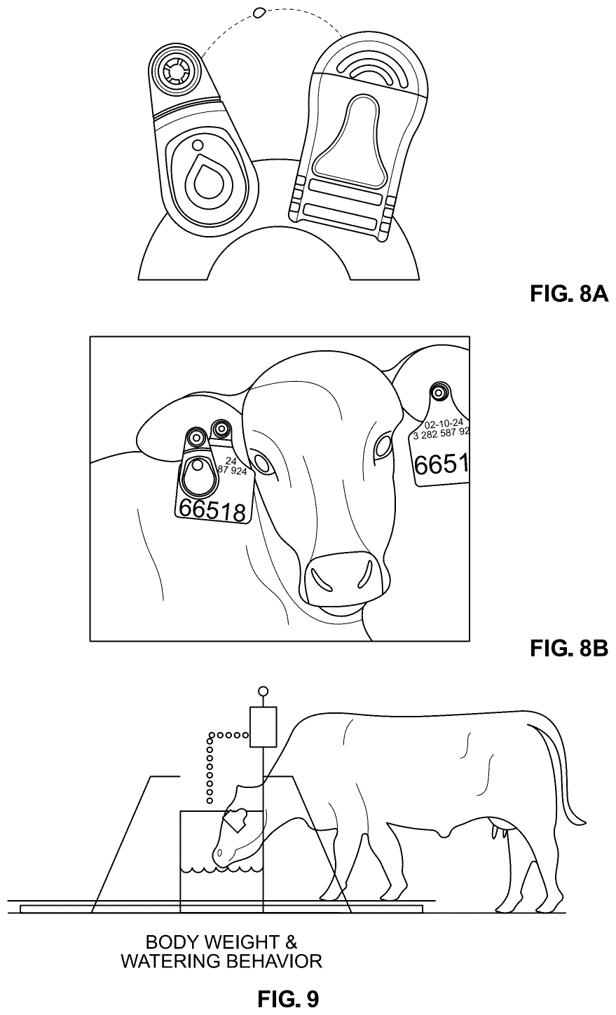

FIGS. 8A-8B are illustrative of the SenseHub™ system where each individual animal in the testing population is fitted with an electronic identification device applied to each animal's ear. The animal is weighed and placed in a pen in which there is a machine able to measure weight. Embodiments of this machine in accordance with the present invention include the commercially-available C-Lock SmartScale™ machine or the Vytelle Sense™ machine. SmartScale (FIG. 9) is a wireless scale system that captures animal weight, performance, and behavior each time it drinks water. This data is collected during a few days before the animal is harvested. At harvest time the actual live weight recorded by the SmartScale™ machine is analyzed along with the harvested hot carcass weight to determine the individual animal's dressing percentage or yield. The test is active over a required period of time to collect adequate and accurate data on each individual animal. The analysis of these individual records is used to select animals with favorable traits which may be used further in a breeding program to provide progeny with the desired trait.

### Example 6: Blood Sampling, DNA Extraction and SNP Detection

As briefly summarized as follows, the sample is prepared and sequenced according to known techniques (for example see WO 2005101230A1, the contents of which are incorporated herein in its entirety). Blood samples were collected from each animal at start of the feed intake test from which genomic DNA was extracted using a modified saturated salt phenol/chloroform procedure (Sambrook et al., Molecular Cloning; A Laboratory Manual 2d ed. (1989)). Identification of polymorphisms in the bovine 1 leptin promoter utilized sequence data with GenBank accession number AB070368. Genomic DNA from a panel of 16 animals was amplified by polymerase chain reaction using forward and reverse primers designed to cover the entire bovine leptin promoter region and the PCR products from each animal were sequenced. Sequence data for each animal were analyzed to identify putative single nucleotide polymorphisms.

A subset of the genotyped animals was sequenced across each polymorphism and the sequence results were used to confirm the genotypes obtained by discrimination assays. In addition to the experimental herd, a total of 160 animals from five commercial lines of relatively unrelated cattle (BeefBooster genetic selection lines MI, M2, M3, M4, and TX) were also genotyped and the allele frequencies of the SNPs were determined in these animals. Foundation breed(s) were Angus for MI, Hereford for M2, various small breeds for M3, Limousin and Gelbvieh for M4, and Charolais for TX (Kress et ah, (1996) J. Anim. Sci. 74:2344-2348).

Chi-square tests were used to examine the genotype frequencies of each polymorphism for deviations from Hardy-Weinberg equilibrium for both the experimental and commercial populations. Differences among the various selection lines of the commercial herd in allele frequencies of the polymorphisms were also tested by chi-square analyses using the Categorical Model Procedure of SAS (SAS Institute, Inc., Gary, NC, 1999). Single marker associations were then determined to evaluate the relationship of the different marker genotypes of each marker on serum leptin concentration, growth rate, body weight, feed intake, feed efficiency and ultrasound traits. The data was analyzed using PROC MIXED of SAS (SAS Institute, Inc., Cary, NC, 1999). The statistical model used included fixed effects of marker genotype, test group (one and two) and sex of animal (bull and steer). Animal was fitted as a random effect to account for background genes. Start weight of animal on test, age of dam or age on test were included in the model as linear covariates. The model used to analyze the carcass data was similar to that of the live animal data but excluded the fixed effects of sex as only steers were sent to slaughter. Associations between different polymorphisms and carcass quality grade were tested by chi-square analyses using the Categorical Model Procedure of SAS (SAS Institute, Inc., Cary, NC, 1999).

Additive genetic effects were estimated for traits that were or tended to be significantly different (P<0.10) between animals with different polymorphism genotypes. Significant additive genetic (a) effects were computed by subtracting the solution of the estimate for the trait effect of the two homozygote genotypes. We also estimated dominance deviation (d) as the deviation of the CT genotypic value from the midpoint between the TT and CC genotypic values.

What is claimed is:

1. A system for adaptive, multi-level processing of health and performance data from processing levels distributed among at least one computing device, wherein the at least one computing device comprises:
a. an input system;
b. a network adapter;
c. a database comprising data;
d. a processing unit, wherein the processing unit is configured to
i. generate one or more machine learning algorithm,
ii. train the machine learning algorithm with the data in the database,
iii. extract, by the machine learning algorithm, new data from the database,
iv. analyze, by the machine learning algorithm, the new data,
v. correlate, by the machine learning algorithm, the new data,
vi. develop a recommendation for the selection of one or more animals to optimize a trait,
vii. receive feedback,
viii. retrain the machine learning algorithm with feedback, wherein retraining the algorithm provides more accurate recommendations; and
e. an output system.

2. The system according to claim 1, wherein the processing unit comprises a central processing unit (CPU) and, optionally, a graphics processing unit (GPU) or multiple processing units, and connects by means of the network adapter to a network, an input and output system, and a database.

3. The system according to claim 2, wherein the network optionally includes a measurement and interaction device.

4. The system according to claim 1, wherein the input and output system receives and collects data inputs from the network to transmit to the database and sends data outputs from the database to the network.

5. The system according to claim 1, wherein the database comprises data and at least one computer software program having instructions for extracting, categorizing, reading and analyzing information, generating outputs from the information, and transmitting the outputs to the network.

6. The system according to claim 1, wherein the input and output system receives and sends data to and from the network and wherein the data resides in the database, said data selected from the group comprising at least gas emissions data, feed intake data, water intake data, health and behavior data, treatment data, sample data, parentage data, measurement data, personalized animal data, clinical and veterinary guidelines, published research data, and genomic data.

7. The system according to claim 1, wherein the database further comprises a content module comprising collected and stored content selected from the group consisting of therapeutic exercises, recorded audio programs, recorded video programs, recommended diets, behavioral data, clinical practice guidelines, published research, educational material, peer-to-peer, peer-to-clinician, and self-to-self messages, surveys, genomic results, animal pedigree and phenotypic data and combinations thereof.

8. The processing unit according to claim 1, further comprising one or more software programs or algorithms having instructions for extracting, categorizing, reading and analyzing information including but not limited to GWAS, SNP traits associations, and genetic evaluation, generating outputs, and transmitting those outputs to the network for action, including but not limited to selection and breeding decisions.

9. The processing unit according to claim 8, wherein the algorithms are selected from the group comprising classification algorithms, recommendation algorithms, analysis algorithms, comparison algorithms, and combinations thereof.

10. The processing unit according to claim 8, wherein the algorithms further include machine learning algorithms, artificial intelligence algorithms, and combinations thereof.

11. The system according to claim 1 wherein the processing unit comprises one or more processing devices selected from the group consisting of a microprocessor, a central processing unit, a complex instruction set computing (CISC) microprocessor, a reduced instruction set computing (RISC) microprocessor, a very long instruction word (VLIW) microprocessor, a processor implementing other instruction sets, a processor implementing a combination of instruction sets, an application specific integrated circuit (ASIC), a field programmable gate array (FPGA), a digital signal processor (DSP), a network processor and combinations thereof.

12. The system according to claim 1, wherein the processing unit communicates with the output system configured to periodically communicate with a user of the system through the measurement and interaction device to provide to the user information and recommendations selected from the group consisting of lifestyle modification recommendations, coaching, therapeutic exercises, recorded audio programs, recorded video programs, behavioral data, clinical practice guidelines, reminders, educational material, advice, and combinations thereof.

13. The system according to claim 1, wherein the computing device is connected through the Internet network.

14. The system according to claim 1, in which the at least one computing device and measurement and interaction device is a cell phone, smartphone, tablet, personal computer, or combination thereof.

15. A method for developing a personalized program for the identification and breeding of animals of superior genetic merit for a variety of traits including but not limited to resistance to Bovine Respiratory Disease, feed efficiency, methane production, embryonic lethality, tenderness and other measures of growth, carcass composition, fertility, longevity and similarly desirable traits, comprising:
a. collecting personal information and other data about the animal through a computing device having an input system and a measurement and interaction device through a network;
b. transferring the collected information to a processing unit, said processing unit further comprising a database for data storage and a plurality of algorithms for analyzing the data and wherein the processing unit is in communication with the input system, the database, and the network;
c. analyzing the information collected in the processing unit using one or more machine learning algorithms to generate at least one set of personalized results and recommendations relevant to the animal and its selection and breeding, including prescribed matings of specific animals, use of conventional and enhanced breeding techniques to propagate superior genetic material in a multi-tiered integrated production system, phenotypic and genomic observations including genotypes and genomic sequence data and other management and breeding methods specific to the animal;
d. storing the results, recommendations, and associated data in the database in communication with an output system;
e. communicating information including at least one personalized set of results and recommendations to the animal through an output system and a measurement and interaction device via a network;
f. receiving feedback;
g. retraining the machine learning algorithm with feedback, wherein retraining the algorithm provides more accurate recommendations.

16. The method according to claim 15, wherein the method further comprises
a. identifying superior animals wherein the superior animals are nucleus or Seedstock Herd animals;
b. using enhanced reproductive technologies to multiply superior genetics;
c. assessing genetic merit using traditional and genomic measures;
d. identifying superior animals for multiplication in Multiplier Herds;
e. designating progeny of the superior animals as commercial cow-calf herds.

17. The method of claim 15, wherein the method further comprises using commercial cow-calf herds to breed stocker or background herds which are sent to feedlot-finishing operations.

18. The method according to claim 16, wherein the genomic measures comprise generating a phenotypic profile of an animal comprising a genotype of the animal defined by at least one single nucleotide polymorphism (SNP) that predict at least one physical characteristic of the animal.

19. The method according to claim 18, wherein the genotype of the animal is further defined by a second panel or a plurality of panels, each panel comprising at least one SNP predicting a physical characteristic of the animal.
